(19) United States
(12) Patent Application Publication
(10) Pub. No.: US 2023/0349898 A1
(43) Pub. Date: Nov. 2, 2023

(54) Methods and Systems for Determining Kinetic Parameters

(71) Applicant: Genentech, Inc., South San Francisco, CA (US)

(72) Inventor: John Gerard Quinn, Belmont, CA (US)

(21) Appl. No.: 18/349,015

(22) Filed: Jul. 7, 2023

Related U.S. Application Data
(63) Continuation of application No. PCT/US22/11632, filed on Jan. 7, 2022.
(60) Provisional application No. 63/135,363, filed on Jan. 8, 2021.

(51) Int. Cl. G01N 33/557; G01N 21/552

(52) U.S. Cl. CPC G01N 33/557; G01N 21/553

(57) ABSTRACT

Methods and systems for determining kinetic parameters are provided. The methods and systems may use a surface having a matrix attached thereto and probe molecules bound to the matrix. Target molecules may be introduced and bind reversibly to the probe molecules. As target molecules are introduced, a first amount of intermolecular complex is generated between the target molecules and the probe molecules and monitored. Once a threshold first amount intermolecular complex is exceeded, introduction of the target molecules may cease. At this point, competitive inhibitor molecules may be introduced and bind to free target molecules produced from continued dissociation of the intermolecular target-probe complex. An amount of the first intermolecular complex may be monitored. This amount may be indicative of the kinetics of a second intermolecular complex between the free target molecules and the competitive inhibitor molecules. In this manner, kinetic parameters of the second intermolecular complex may be estimated.

### PRIORITY

This application claims the benefit under 35 U.S.C. § 365(c) of International Patent Application No. PCT/US2022/011632, filed 7 Jan. 2022, which claims the benefit under 35 U.S.C. § 119(e) of U.S. Provisional Patent Application No. 63/135,363, filed 8 Jan. 2021, each of which is incorporated herein by reference in its entirety.

### TECHNICAL FIELD

This disclosure generally relates to assay methods and systems, and in particular relates to assay methods and systems for estimating kinetic parameters of molecules.

### BACKGROUND

There is a need for pharmaceuticals, biologics, drugs, or other therapeutics that target macromolecules for which the development of such therapeutics has been difficult. Some of these macromolecules form transient complexes with therapeutics. However, current systems and methods may be unable to determine the dynamics of such transient complexes, making it difficult to identify suitable lead compounds for therapeutics targeting these macromolecules. As such, there is a need for methods and system for determining kinetic parameters for short-lived macromolecular-therapeutic complexes and for extremely rapid association of such complexes.

### SUMMARY OF PARTICULAR EMBODIMENTS

In an aspect, the present disclosure provides an assay method for estimating a kinetic parameter, comprising: (a) introducing a plurality of target molecules to a first location, the first location comprising: (i) a first surface, (ii) a matrix bound to the first surface, and (iii) a plurality of probe molecules bound to the matrix; (b) monitoring, via a detection method, a first amount of an intermolecular complex generated by the plurality of probe molecules and the plurality of target molecules; (c) when the first amount exceeds a threshold value, stopping the introduction of the plurality of target molecules and introducing a plurality of competitive inhibitor molecules to the first location; (d) monitoring, via the detection method, a second amount of the intermolecular complex; and (e) estimating the kinetic parameter based upon the second amount. In various embodiments, the kinetic parameter comprises an association rate constant between the plurality of target molecules and the plurality of competitive inhibitor molecules. In various embodiments, the kinetic parameter comprises a dissociation rate constant between the plurality of target molecules and the plurality of competitive inhibitor molecules. In various embodiments, the first location comprises a first channel in a flow cell. In various embodiments, (a) comprises: starting a flow of the plurality of target molecules through the first channel and (c) comprises: when the first amount exceeds a threshold value, stopping the flow of the plurality of target molecules through the first channel and starting a flow of the plurality of competitive inhibitor molecules through the first channel. In various embodiments, the assay method further comprises monitoring, via the detection method, a second location comprising a second channel in a flow cell, wherein the second location comprises a second surface. In various embodiments, the second location does not comprise a probe molecule bound to at least the second surface. In various embodiments, the first location comprises a first well in a well plate. In various embodiments, (a) comprises: placing the plurality of target molecules into the first well and (c) comprises: when the first amount exceeds a threshold value, stopping the placement of the plurality of target molecules into the first well and placing the plurality of competitive inhibitor molecules into the first well. In various embodiments, the assay method further comprises monitoring, via the detection method, a second location comprising a second well in a well plate, wherein the second location comprises (i) a surface and (ii) a matrix bound to the surface. In various embodiments, the second location does not comprise a matrix. In various embodiments, the second location does not comprise a plurality of probe molecules. In various embodiments, the matrix comprises a hydrogel. In various embodiments, the kinetic parameter is within a predetermined range and one or more dimensions of the matrix are chosen to sensitize the method to the predetermined range. In various embodiments, the one or more dimensions of the matrix adre within a range from 5 nanometers (nm) to 1,000 nm. In various embodiments, the kinetic parameter is within a predetermined range and a density of the plurality of probe molecules is chosen to sensitize the method to the predetermined range. In various embodiments, the detection method comprises a surface plasmon resonance (SPR) method. In various embodiments, the first amount of the intermolecular complex is generated from reversible affinity interactions between the plurality of probe molecules and the plurality of target molecules. In various embodiments, (b) comprises, for a plurality of points in time: detecting, via the detection method, a detection signal indicative of the first amount. In various embodiments, (c) comprises, for a plurality of points in time: (i) comparing the first amount with the threshold value; and (ii) if the first amount exceeds the threshold value, stopping the introduction of the plurality of target molecules and introducing the plurality of competitive inhibitor molecules to the first location. In various embodiments, the first amount increases over the plurality of points in time. In various embodiments, (d) comprises, for a plurality of points in time: detecting, via the detection method, a detection signal indicative of the second amount. In various embodiments, the second amount decreases over the plurality of points in time. In various embodiments, the assay method further comprises, prior to (a), binding the matrix to the first surface and binding the plurality of probe molecules to the matrix.

In another aspect, the present disclosure provides an assay system for estimating a kinetic parameter, comprising: (a) a flow manifold capable of fluidically coupling to an assay module, the assay module comprising: (i) a first location comprising a first surface, (ii) a matrix bound to the first surface, and (iii) a plurality of probe molecules bound to the matrix; (b) a detector; and (c) a controller configured to: (i) direct the flow manifold to introduce a first solution comprising the plurality of target molecules to the first location; (ii) direct the detector to monitor a first amount of an intermolecular complex generated by the plurality of probe molecules and the plurality of target molecules; (iii) direct the flow manifold to, when the first amount exceeds a threshold value, stop the introduction of the first solution and introduce a second solution comprising a plurality of competitive inhibitor molecules to the first location; (iv) direct the detector to monitor a second amount of the intermolecular complex; and (v) determine the kinetic parameter based upon the second amount. In various embodiments, the kinetic parameter comprises an association rate constant between the plurality of target molecules and the plurality of competitive inhibitor molecules. In various embodiments, the kinetic parameter comprises a dissociation rate constant between the plurality of target molecules and the plurality of competitive inhibitor molecules. In various embodiments, the first location comprises a first channel in a flow cell. In various embodiments, (c)(i) comprises: starting a flow of the plurality of target molecules through the first channel and (c)(iii) comprises: when the first amount exceeds a threshold value, stopping the flow of the plurality of target molecules through the first channel and starting a flow of the plurality of competitive inhibitor molecules through the first channel. In various embodiments, the controller is further configured to direct the detector monitor a second location comprising a second channel in a flow cell, wherein the second location comprises a second surface. In various embodiments, the second location does not comprise a probe molecule bound to at least the second surface. In various embodiments, the first location comprises a first well in a well plate. In various embodiments, (c)(i) comprises: placing the plurality of target molecules into the first well and (c)(iii) comprises: when the first amount exceeds a threshold value, stopping the placement of the plurality of target molecules into the first well and placing the plurality of competitive inhibitor molecules into the first well. In various embodiments, the controller is further configured to direct the detector to monitor a second location comprising a second well in a well plate, wherein the second location comprises (i) a surface and (ii) a matrix bound to the surface. In various embodiments, the second location does not comprise a matrix. In various embodiments, the second location does not comprise a plurality of probe molecules. In various embodiments, the matrix comprises a hydrogel. In various embodiments, the kinetic parameter is within a predetermined range and one or more dimensions of the matrix are chosen to sensitize the method to the predetermined range. In various embodiments, the one or more dimensions of the matrix are within a range from 5 nm to 1,000 nm. In various embodiments, the kinetic parameter is within a predetermined range and a density of the plurality of probe molecules is chosen to sensitize the method to the predetermined range. In various embodiments, the detector comprises a surface plasmon resonance (SPR) detector. In various embodiments, the first amount of the intermolecular complex is generated from reversible affinity interactions between the plurality of probe molecules and the plurality of target molecules. In various embodiments, (c)(ii) comprises, for a plurality of points in time: detecting a detection signal indicative of the first amount. In various embodiments, (c)(iii) comprises, for a plurality of points in time: (i) comparing the first amount with the threshold value; and (ii) if the first amount exceeds the threshold value, stopping the introduction of the plurality of target molecules and introducing the plurality of competitive inhibitor molecules to the first location. In various embodiments, the first amount increases over the plurality of points in time. In various embodiments, (c)(iv) comprises, for a plurality of points in time: detecting a detection signal indicative of the second amount. In various embodiments, the second amount decreases over the plurality of points in time.

In another aspect, the present disclosure provides an assay module for estimating a kinetic parameter, comprising: (i) a first location comprising a first surface; (ii) a matrix bound to the first surface; and (iii) a plurality of probe molecules bound to the matrix and configured to form an intermolecular complex with a plurality of target molecules, wherein the target molecules escape the intermolecular complex and the first location at a predetermined rate, the rate based on a thickness of the matrix, a density of the matrix, an extent of crosslinking of the matrix, a viscosity of the matrix, or a density of the plurality of probe molecules. In various embodiments, the first location comprises a first channel in a flow cell. In various embodiments, the assay module further comprises a second location comprising a second channel in a flow cell, wherein the second location comprises a second surface. In various embodiments, the second location does not comprise a probe molecule bound to at least the second surface. In various embodiments, the first location comprises a first well in a well plate. In various embodiments, the assay module further comprises a second location comprising a second well in a well plate, wherein the second location comprises (i) a surface and (ii) a matrix bound to the surface. In various embodiments, the second location does not comprise a matrix. In various embodiments, the second location does not comprise a plurality of probe molecules. In various embodiments, the matrix comprises a hydrogel. In various embodiments, the one or more dimensions of the matrix are within a range from 5 nm to 1,000 nm.

In another aspect, the present disclosure provides a kit for conducting an assay for estimating a kinetic parameter, comprising: (a) an assay module comprising a first location comprising (i) a first surface, (ii) a matrix bound to the first surface, and (iii) a plurality of probe molecules bound to the matrix; (b) a first solution comprising a plurality of target molecules capable of forming a first intermolecular complex with the plurality of probe molecules; and (c) a second solution comprising a plurality of competitive inhibitor molecules capable of forming a second intermolecular complex with the plurality of target molecules, wherein the composition of the matrix is optimized for diffusion of the target molecule. In various embodiments, the first location comprises a first channel in a flow cell. In various embodiments, the assay module further comprises a second location comprising a second channel in a flow cell, wherein the second location comprises a second surface. In various embodiments, the second location does not comprise a probe molecule bound to at least the second surface. In various embodiments, the first location comprises a first well in a well plate. In various embodiments, the assay module further comprises a second location comprising a second well in a well plate, wherein the second location comprises (i) a surface and (ii) a matrix bound to the surface. In various embodiments, the second location does not comprise a matrix. In various embodiments, the second location does not comprise a plurality of probe molecules. In various embodiments, the matrix comprises a hydrogel. In various embodiments, the one or more dimensions of the matrix are within a range from 5 nm to 1,000 nm.

In another aspect, the present disclose provides an assay method for estimating a kinetic parameter, comprising: (a) introducing a plurality of target molecules to a first location, the first location comprising: (i) a first surface, (ii) a matrix bound to the first surface, and (iii) a plurality of probe molecules bound to the matrix and configured to form an intermolecular complex with the plurality of target molecules; (b) stopping the introduction of the plurality of target molecules and introducing a plurality of competitive inhibitor molecules to the first location; (c) monitoring, via a detection method, an amount of the intermolecular complex; and (d) estimating the kinetic parameter based upon the amount.

### BRIEF DESCRIPTION OF THE DRAWINGS

FIG. 1 is a simplified diagram of a rebinding assay for determining a kinetic parameter, in accordance with various embodiments.

FIG. 2 is a simplified diagram of rebinding curves for determining a kinetic parameter, in accordance with various embodiments.

FIG. 3 is a simplified diagram of a method for determining a kinetic parameter, in accordance with various embodiments.

FIG. 4 is a block diagram of a system for determining a kinetic parameter, in accordance with various embodiments.

FIG. 5 is a block diagram of a channel-based assay module for determining a kinetic parameter, in accordance with various embodiments.

FIG. 6 is a block diagram of a well-based assay module for determining a kinetic parameter, in accordance with various embodiments.

FIG. 7 is a block diagram of a functionalized surface, in accordance with various embodiments.

FIG. 8 is a block diagram of a computer-based system for determining a kinetic parameter, in accordance with various embodiments.

FIG. 9 is a block diagram of a computer system, in accordance with various embodiments.

FIG. 10 is a block diagram of a kit for estimating a kinetic parameter, in accordance with various embodiments.

FIG. 11A shows upper and lower limit response curves corresponding to zero inhibition and full inhibition, in accordance with various embodiments.

FIG. 11B shows relative errors associated with the data of FIG. 11A, in accordance with various embodiments.

FIG. 11C shows confidence intervals associated with the data of FIG. 11A, in accordance with various embodiments.

FIG. 12A shows conventional surface plasmon resonance (SPR) curves for interaction of soluble probe molecules with hydrogel-bound target molecules, in accordance with various embodiments.

FIG. 12B shows conventional SPR curves for interaction of soluble target molecules with hydrogel-bound probe molecules, in accordance with various embodiments.

FIG. 12C shows estimation of parameters from a target capture region of the rebinding assay curves of FIGS. 12A and 12B, in accordance with various embodiments.

FIG. 12D shows inhibition curves for the rebinding assay of FIG. 12C, in accordance with various embodiments.

FIG. 13A shows inhibition curves corresponding to a variety of association rate constants, in accordance with various embodiments.

FIG. 13B shows partition curves for loss of inhibition of rebinding as a function of transient dissociation rate constant, in accordance with various embodiments.

FIG. 13C shows correlation of fitted dissociation rate constants with true dissociation rate constants for the data shown in FIG. 13A, in accordance with various embodiments.

FIG. 13D shows correlation of fitted association rate constants with true dissociation rate constants for the data shown in FIG. 13B, in accordance with various embodiments.

FIG. 13E shows divergence of response-normalized dissociation curves over a range of SPR response values, in accordance with various embodiments.

FIG. 13F shows relative error in kinetic parameters returned from fitting sets of inhibition curves corresponding to a wide range in surface saturation, in accordance with various embodiments.

FIG. 14A shows an affinity space plot for a competitive kinetics assay with contour curves connecting regions of equal response, in accordance with various embodiments.

FIG. 14B shows an affinity plot space for the rebinding assays described herein, in accordance with various embodiments.

FIG. 14C shows correlation of true association rate constants with values estimated from fitting the competitive kinetic model.

FIG. 14D shows correlation of true association rate constants with values estimated from fitting the competitive kinetic model when restricting the range of dissociation rate constants, in accordance with various embodiments.

FIG. 14E shows the data of FIG. 14D given in terms of true dissociation rate constants versus fitted dissociation rate constants, in accordance with various embodiments.

FIG. 14F shows correlation of true association rate constants with values estimated from fitting the rebinding models described herein, in accordance with various embodiments.

FIG. 14G shows correlation of true dissociation rate constants with values estimated from fitting the rebinding models described herein, in accordance with various embodiments.

In various embodiments, not all of the depicted components in each figure may be required, and various embodiments may include additional components not shown in a figure. Variations in the arrangement and type of the components may be made without departing from the scope of the subject disclosure. Additional components, different components, or fewer components may be utilized within the scope of the subject disclosure. In the figures, like numbers denote like elements.

### DESCRIPTION OF EXAMPLE EMBODIMENTS

This specification describes exemplary methods and systems for determining kinetic parameters (for instance, for short-lived macromolecular-therapeutic complexes).

The methods and systems may generally operate by using a surface (such as a noble metal surface used for a surface plasmon resonance (SPR) measurement) having probe molecules attached thereto. Target molecules may be introduced and may bind to the probe molecules. The target molecules may comprise a therapeutic target, such as a peptide, polypeptide, protein, protein complex, nucleic acid, histone, or another other macromolecule of interest. The term “target” is used herein in the most general sense and may be any affinity binding partner capable of forming an affinity complex with the probe molecules. The term “target” should not be interpreted as being confined to meaning a species that is being targeted for therapeutic intervention.

As the target molecules are introduced, a first intermolecular complex between the target molecules and the probe molecules may be formed. The formation of this first intermolecular complex may be monitored by a detection method, such as an SPR detection method. Once a threshold amount of the first intermolecular complex is formed, introduction of the target molecules may cease and dissociation of the first intermolecular complex may commence. At this point, competitive inhibitor molecules may be introduced and may form a second intermolecular complex by binding to unbound target molecules remaining at the surface through multiple cycles of unbinding and/or rebinding that are promoted by a high concentration of bound probe molecules. An amount of the first intermolecular complex may once again be monitored by the detection method. This amount may be indicative of the kinetics of the second intermolecular complex between the target molecules and the competitive inhibitor molecules. In this manner, kinetic parameters (such as association rate constants or dissociation rate constants) of the second intermolecular complex may be estimated.

The methods and systems may have particular utility in the identification of early chemical matter that may then be optimized to generate lead compounds for therapeutics having kinetic parameters within a range of interest. The methods and systems may have particular utility in characterizing kinetic parameters that are difficult to measure using current methods. For instance, the methods and system may be useful in measuring association rate constants and/or dissociation rate constants for transient macromolecular complexes that typically dissociate less than a few hundred milliseconds (ms) after formation. In addition, the methods and system may be particularly useful in measuring association rate constants for affinity complexes that form rapidly (e.g., when kₐ>1×10⁷ M⁻¹s⁻¹) and dissociate in greater than a few hundred milliseconds. Such complexes may include indefinitely stable complexes that are considered fully irreversible.

As used herein, the term “or” shall be interpreted as conveying both exclusive and inclusive meaning. For instance, reference to elements A or B shall be interpreted as disclosing element A alone, element B alone, or the combination of elements A and B.

Determining a Kinetic Parameter from a Rebinding Assay

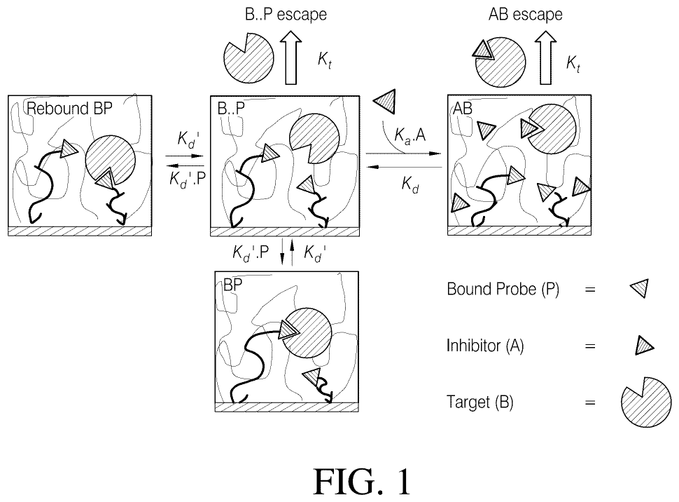

FIG. 1 is a simplified diagram of a rebinding assay for determining a kinetic parameter. The process depicted in FIG. 1 starts after the threshold amount of the first intermolecular complex between the target molecules and the probe molecules has been formed. As shown in panel (i) of FIG. 1, probe molecules P are bound to a surface and optionally to a matrix in which the probe molecules P are attached (bound or tethered). A first intermolecular complex BP between the probe molecules and the target molecules B is formed during the introduction of the target molecules. When the introduction of the target molecules is stopped, the first intermolecular complex BP may dissociate to form free probe molecules P and free target molecules B. This process may occur with dissociation rate constant k_{d}′. The probe molecules P may remain tethered to the surface either directly to a barrier layer on the surface or to a matrix film attached to the barrier layer. The target molecules B are not tethered to the surface or to the matrix and in the absence of any other interactions within the sensing layer will escape the matrix with rate constant kₜ, as shown in panel (ii) of FIG. 1. However, the probe molecules P and target molecules B may also re-form into the first intermolecular complex BP through a rebinding process, as shown in panel (iii) of FIG. 1. This rebinding process may occur with association rate constant k_{d}′*[P] (with [P] the concentration of the probe molecules P) and dissociation rate constant k_{d}′. This rebinding process may result in a decrease in the measured apparent dissociation rate constant k_{off} of the first intermolecular complex relative to the true dissociation rate constant k_{d}′.

The rebinding process may be decreased (in some cases, decreased substantially or nearly negated) by introducing competitive inhibitor molecules A immediately following stopping of the introduction of target molecules B. As shown in panel (iv) of FIG. 1, the introducing of the inhibitor molecules A may cause formation of an inhibition complex AB (also referred to as a second intermolecular complex herein). The inhibition complex AB may nearly restore the measured apparent dissociation rate constant, such that k_{off}≅k_{d}′. The inhibition complex AB may escape the matrix with rate constant kₜ. The dynamics of the system described in FIG. 1 may be written as a series of coupled differential equations:

$$\frac{d [ B ]}{dt} = - {k}_{a} * [ A ] * [ B ] + {k}_{d} * [ AB ] - {k}_{a}^{′} * [ B ] * [ P ] + {k}_{d}^{′} * [ BP ] - \frac{{k}_{t}}{G * {M}_{{r}_{B}}} * ( [ B ] + [ AB ] ) \qquad ( 1 )$$

$$\frac{d [ A B ]}{dt} = {k}_{a} * [ A ] * [ B ] - {k}_{d} * [ A B ] - \frac{{k}_{t}}{G * {M}_{{r}_{B}}} * [ AB ] \qquad ( 2 )$$

$$\frac{d [ P ]}{dt} = - {k}_{a}^{′} * [ B ] * [ P ] + {{k}_{d}}^{′} * [ B P ] \qquad ( 3 )$$

$$\frac{d [ B P ]}{dt} = {k}_{a}^{′} * [ B ] * [ P ] - {{k}_{d}}^{′} * [ B P ] \qquad ( 4 )$$

In Equations (1)-(4), kₐ and k_{d} are the association and dissociation rate constants, respectively, for the second intermolecular complex AB and kₐ′ and k_{d}′ are the association and dissociation rate constants, respectively, for the first intermolecular complex BP. [A], [B], [AB], [P], and [BP] are the concentrations of the inhibitor molecules A, the target molecules B, the second intermolecular complex BP, the probe molecules P, and the first intermolecular complex BP, respectively. M_{r_B} is the molecular weight of B. The four coupled partial differential equations given by Equations (1)-(4) may be modified to express a biosensor output signal as a measure of affinity complex accumulation, as described in Equations (12)-(20) herein. Alternatively or in combination, an approximate algebraic model described herein may be fit to experimental data, as described herein.

The flux balance between the surface reaction and mass transport limitation may be expressed by the dimensionless Damköhler number Dₐ=kₐ′*(Rₘₐₓ−R)/k′ₜ, where Rₘₐₓ is the detector (for instance, an SPR detector, as described herein) response at saturation, R is the response at any given time, and k′ₜ is the combined mass transport constant and has components related to convective transport and diffusive resistance within the matrix. The combined mass transport constant may be written as:

k′ₜ=kₜ*M_{r_B}*G (5)

Here,

$${k}_{t} = {T}_{γ} * 1.281 * \sqrt[3]{\frac{{v}_{c} * D}{2 * h * l}} ,$$

and G is the response-to-concentration factor and expresses Rₙₐₓ as a molar concentration of P. G may be chosen to equal 100 on the assumption that 1 gram per liter of protein distributed homogeneously within the matrix produces a detector response of 100 RU or may be experimentally determined for any particular detector. D is the bulk diffusion coefficient of the second intermolecular complex, h is the height of the location within which the assay is conducted (such as a flow channel or well, as described herein), l is the length of the location within which the assay is conducted, and v_{c} is the maximum flow velocity in the location within which the assay is conducted (such as the in the center of the flow channel or well). The incorporation of the matrix transport resistance term T_{γ} accounts for matrix transport resistance and is defined in terms of the height of the matrix Hₘₐₜᵣᵢₓ relative to the mean free path taken by B before being bound:

$${T}_{γ} = \frac{tan h ( γ )}{γ} .$$

Here, γ=Hₘₐₜᵣᵢₓ*(Dₘₐₜᵣᵢₓ*Kₚₐᵣₜ/k′ₐ[P])^{-0.5}, where Kₚₐᵣₜ is the unitless matrix partition coefficient and Dₘₐₜᵣᵢₓ is the diffusion coefficient of B within the matrix.

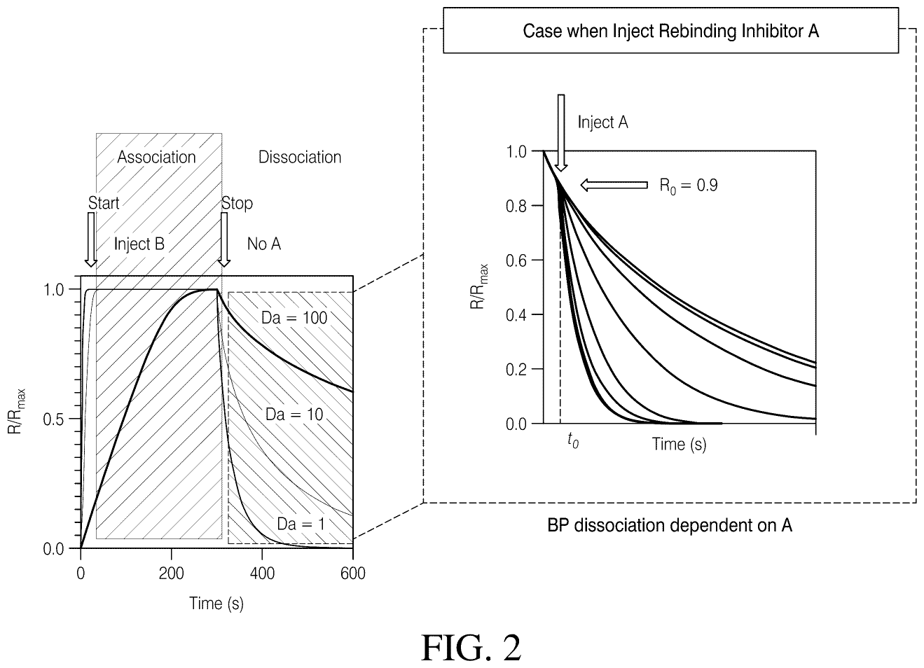

FIG. 2 is a simplified diagram of rebinding curves for determining a kinetic parameter. As shown in FIG. 2, the rebinding curve dynamics following introduction of the competitive inhibitor molecules are highly dependent upon Dₐ, which is itself dependent upon the geometry of the detection system used to obtain the inhibition curves and on the properties of the matrix. Phenomenologically, these dynamics may be modeled by a single decaying exponential, thereby avoiding the need to describe highly complicated and difficult to calculate biophysical parameters. That is, the decaying curve R that results following introduction of the competitive inhibitor molecule may be written as:

R=R₀ exp(−k_{off}*t) (6)

Injection of A may produce an inhibition flux that increases k_{off} by lowering the rebinding flux such that k_{off}≈k′_{d} when fully inhibited. Mesoscopic transition state-based modeling reduces to a flux balance defined by a single rebinding factor α yields R=R₀ exp(−k′_{d}*α*t), which is suitable for estimation of kinetic constants. Here:

$${k}_{off} = {{k}_{d}}^{′} * α \qquad ( 7 )$$

$$α = \frac{β}{β + {k}_{a}^{′} * [ P ]} \qquad ( 8 )$$

$$P = {R}_{max} / ( {M}_{r \_ B} * G ) \qquad ( 9 )$$

$$β = {k}_{t} + f * {k}_{a} * [ A ] \qquad ( 10 )$$

$$f = {( 1 + {k}_{d} / {k}_{t} )}^{- 1} \qquad ( 11 )$$

The rebinding factor α is the degree to which dissociation is slowed due to rebinding and is given by the ratio of the rebinding flux k′ₐ*[P] to the total matrix escape flux β. Thus, α=1 when rebinding does not exist and α<1 otherwise. The partition function ƒ accounts for loss in inhibition of rebinding due to unbinding of AB before escaping the matrix. The rate constants associated with the partition functions are high relative to k′_{d}, allowing quasi-steady-state conditions to be assumed.

Equations (6)-(11) may be locally and/or globally fit to any set of inhibition curves obtained for any set of probe molecules, target molecules, and competitive inhibitor molecules. Such global fitting may allow estimation of the kinetic parameters governing association and dissociation of the first and second intermolecular complexes.

Alternatively or in combination, the experimental data can be modeled and fit to a general model expressed in terms of the ordinary differential equations (ODEs) defined by Equations (1-4) after adding boundary conditions and unit conversion factors that relate concentration to biosensor response. The resulting Equations (12)-(20) may be used in combination with numerical integration and non-linear least squares curve fitting to fit experimental data, as described herein. Assuming there is negligible mass transport resistance for A within the matrix (since [B] will typically remain comparatively low), the coupled set of ODEs is given by:

$$\frac{d [ B ]}{dt} = - {k}_{a} * [ A ] * [ B ] + {k}_{d} * [ AB ] - {k}_{a}^{′} * [ B ] * [ P ] + {k}_{d}^{′} * [ BP ] - \frac{{k}_{t}^{′}}{{M}_{{r}_{B}} * G} * ( [ B ] + [ A B ] ) \qquad ( 12 )$$

$$\frac{d [ A B ]}{dt} = {k}_{a} * [ A ] * [ B ] - {k}_{d} * [ A B ] - \frac{{k}_{t}^{′}}{{M}_{{r}_{B}} * G} * [ AB ] \qquad ( 13 )$$

$$\frac{d [ P ]}{dt} = {k}_{a}^{′} * [ B ] * [ P ] - {{k}_{d}}^{′} * [ B P ] \qquad ( 14 )$$

$$\frac{d [ B P ]}{dt} = {k}_{a}^{′} * [ B ] * [ P ] - {{k}_{d}}^{′} * [ B P ] \qquad ( 15 )$$

$$[ P ] ( {t}_{0} ) = [ {P}_{0} ] / ( {M}_{{r}_{B}} * G ) \qquad ( 16 )$$

$$[ B ] ( {t}_{0} ) = [ {B}_{0} ] / ( {M}_{{r}_{B}} * G ) \qquad ( 17 )$$

$$[ BP ] ( {t}_{0} ) = [ B {P}_{0} ] / ( {M}_{{r}_{B}} * G ) \qquad ( 18 )$$

$$[ AB ] ( {t}_{0} ) = 0 \qquad ( 19 )$$

$$R ( {t}_{0} ) = [ B P ] * {M}_{{r}_{B}} * G \qquad ( 20 )$$

Here, [P₀], [B₀], and [BP₀] are the initial concentrations of P, B, and BP in terms of their sensor responses, respectively. Equations (12)-(20) may be numerically integrated and fitted by a fitting procedure (such as a non-linear least squares fitting procedure or a fitting procedure utilizing any acceptable linear or non-linear loss function) to any set of inhibition curves obtained for any set of probe molecules, target molecules, and competitive inhibitor molecules. Such numerical integration and fitting may allow estimation of the kinetic parameters governing association and dissociation of the first and second intermolecular complexes.

Equations (12)-(20) hold for formation of reversible affinity complexes AB. They can be modified to include formation of a second reversible state (AB*, with concentration [AB*]) described by forward and reverse rate constants k_{f} and kᵣ, respectively. In such cases, Equations (12)-(20) may be replaced by Equations (21)-(31) and solved by similar numerical integration and fitting:

$$\frac{d [ B ]}{dt} = - {k}_{a} * [ A ] * [ B ] + {k}_{d} * [ AB ] - {k}_{a}^{′} * [ B ] * [ P ] + {k}_{d}^{′} * [ B P ] - \frac{{k}_{t}^{′}}{{M}_{{r}_{B}} * G} * ( [ B ] + [ A B ] ) \qquad ( 21 )$$

$$\frac{d [ A B ]}{dt} = {k}_{a} * [ A ] * [ B ] - {k}_{d} * [ A B ] - {k}_{f} * [ A B ] + {k}_{r} * [ A {B}^{*} ] - \frac{{k}_{t}^{′}}{{M}_{{r}_{B}} * G} * [ AB ] \qquad ( 22 )$$

$$\frac{d [ {AB}^{*} ]}{dt} = {k}_{f} * [ A B ] - {k}_{r} * [ A {B}^{*} ] - \frac{{k}_{t}^{′}}{{M}_{{r}_{B}} * G} * [ A {B}^{*} ] \qquad ( 23 )$$

$$\frac{d [ P ]}{dt} = - {k}_{a}^{′} * [ B ] * [ P ] + {{k}_{d}}^{′} * [ B P ] \qquad ( 24 )$$

$$\frac{d [ B P ]}{dt} = {k}_{a}^{′} * [ B ] * [ P ] - {{k}_{d}}^{′} * [ B P ] \qquad ( 25 )$$

$$[ P ] ( {t}_{0} ) = [ {P}_{0} ] / ( {M}_{{r}_{B}} * G ) \qquad ( 26 )$$

$$[ B ] ( {t}_{0} ) = [ {B}_{0} ] / ( {M}_{{r}_{B}} * G ) \qquad ( 27 )$$

$$[ BP ] ( {t}_{0} ) = [ B {P}_{0} ] / ( {M}_{{r}_{B}} * G ) \qquad ( 28 )$$

$$[ AB ] ( {t}_{0} ) = 0 \qquad ( 29 )$$

$$[ {AB}^{*} ] ( {t}_{0} ) = 0 \qquad ( 30 )$$

$$R ( {t}_{0} ) = [ B P ] * {M}_{{r}_{B}} * G \qquad ( 31 )$$

The model described by Equations (21)-(31) may be applied when binding of A to B is driven only by affinity interactions with reversible conformational changes, or when a reversible covalent adduct forms as the second state.

The inhibition curves may be similar in form to those shown in FIG. 2. This may be true for a wide range in fractional occupancy over a wide range in Dₐ and is fundamentally dependent on the kinetics of all interactants. However, as shown in Equation (5), the inhibition curves are also dependent upon the geometry of the microfluidic channels and flow rate. Equation (5) also shows that the sensing environment used to obtain the inhibition curves governs fundamental processes such as diffusion which in turn affect all kinetic rates. For example, the diffusion rate constant for the second intermolecular complex within the matrix has a large impact on kₜ and this rate constant defines the kinetic regimes governing the assay described in FIG. 1 and FIG. 2. This diffusion is dependent upon properties of the matrix, such as matrix thickness, density, extent of crosslinking, viscosity and so forth. Moreover, as implied by Equations (5)-(11), (12)-(20), and (21)-(31), the method may be optimized by generating sufficient rebinding potential (as defined by a) to produce a large difference between the fully inhibited and uninhibited rebinding curves. However, this must be balanced against sensitivity since sensitivity, or vulnerability to inhibition, is more optimum at lower a. As such, the method can be sensitized to detect kinetic parameters within a range of interest based upon some combination of the parameters in Equations (6)-(11), (12)-(20), and/or (21)-(31), which yield acceptable results for a wide range of parameter combinations. Such flexibility in acceptable parameter combinations may provide compensation for non-ideal constraints that may be more difficult to alter, such as the kinetic rate constants associated with the probe/target and inhibitor/target interaction complexes.

### Methods for Determining a Kinetic Parameter

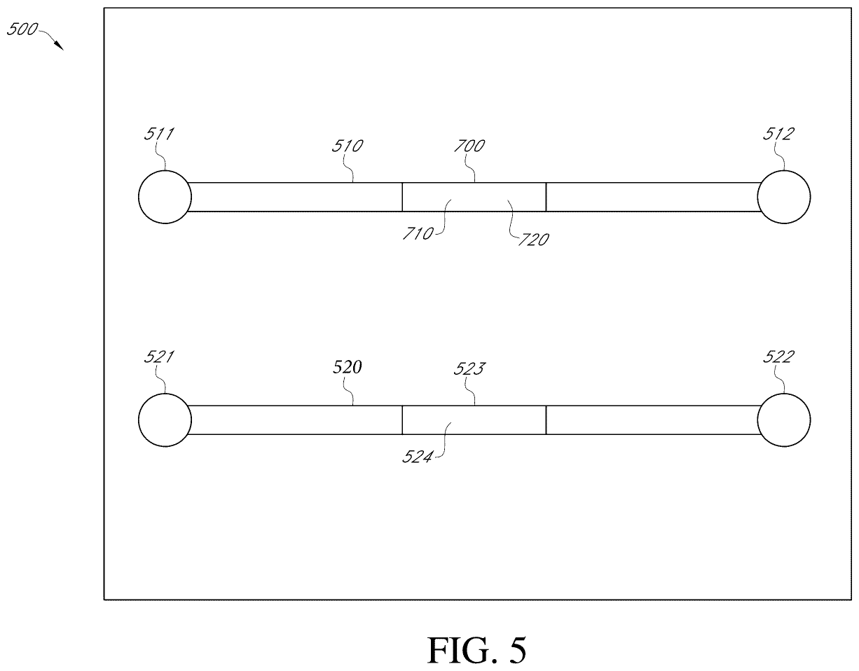

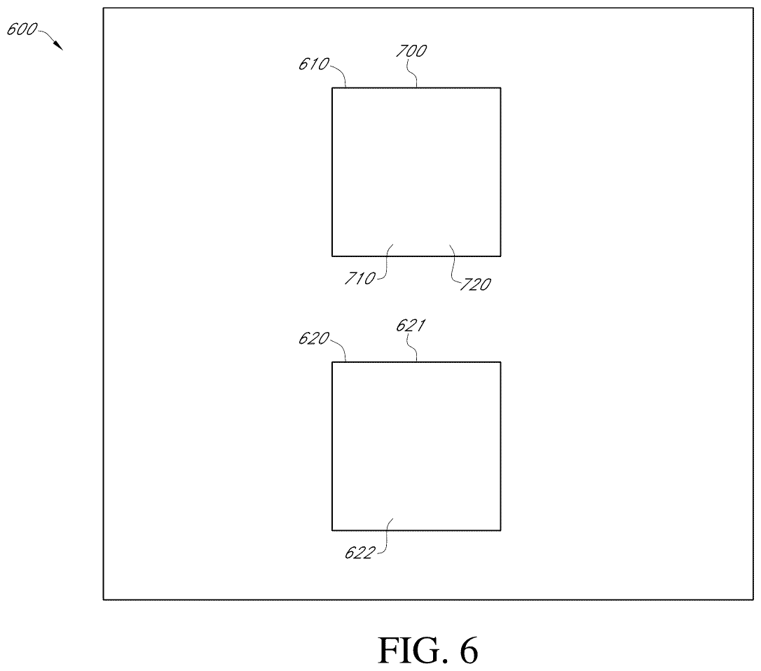

In accordance with the principles discussed herein with respect to FIGS. 1 and 2, FIG. 3 is a simplified diagram of a method 300 for determining a kinetic parameter. In various embodiments, the method 300 may comprise a first step 310 of introducing a plurality of target molecules to a first location. The first location may comprise a first channel in a flow cell, as described herein with respect to FIG. 5. The first location may comprise a first well in a well plate, as described herein with respect to FIG. 6.

In various embodiments, the plurality of target molecules comprises a therapeutic target, such as a peptide, polypeptide, protein, protein complex, nucleic acid, histone, chromosome, or another other macromolecule of interest, as described herein. The plurality of target molecules may be similar to the target molecules B described herein with respect to FIGS. 1 and 2. The plurality of target molecules may comprise any number of target molecules. For instance, the plurality of target molecules may comprise at least about 1 femtomole (fmol), 2 fmol, 3 fmol, 4 fmol, 5 fmol, 6 fmol, 7 fmol, 8 fmol, 9 fmol, 10 fmol, 20 fmol, 30 fmol, 40 fmol, 50 fmol, 60 fmol, 70 fmol, 80 fmol, 90 fmol, 100 fmol, 200 fmol, 300 fmol, 400 fmol, 500 fmol, 600 fmol, 700 fmol, 800 fmol, 900 fmol, 1 picomole (pmol), 2 pmol, 3 pmol, 4 pmol, 5 pmol, 6 pmol, 7 pmol, 8 pmol, 9 pmol, 10 pmol, 20 pmol, 30 pmol, 40 pmol, 50 pmol, 60 pmol, 70 pmol, 80 pmol, 90 pmol, 100 pmol, 200 pmol, 300 pmol, 400 pmol, 500 pmol, 600 pmol, 700 pmol, 800 pmol, 900 pmol, 1 nanomole (nmol), 2 nmol, 3 nmol, 4 nmol, 5 nmol, 6 nmol, 7 nmol, 8 nmol, 9 nmol, 10 nmol, 20 nmol, 30 nmol, 40 nmol, 50 nmol, 60 nmol, 70 nmol, 80 nmol, 90 nmol, 100 nmol, 200 nmol, 300 nmol, 400 nmol, 500 nmol, 600 nmol, 700 nmol, 800 nmol, 900 nmol, 1 micromole (μmol), 2 μmol, 3 μmol, 4 μmol, 5 μmol, 6 μmol, 7 μmol, 8 μmol, 9 μmol, 10 μmol, 20 μmol, 30 μmol, 40 μmol, 50 μmol, 60 μmol, 70 μmol, 80 μmol, 90 μmol, 100 μmol, 200 μmol, 300 μmol, 400 μmol, 500 μmol, 600 μmol, 700 μmol, 800 μmol, 900 μmol, 1,000 μmol, or more of target molecules. The plurality of target molecules may comprise at most about 1,000 μmol, 900 μmol, 800 μmol, 700 μmol, 600 μmol, 500 μmol, 400 μmol, 300 μmol, 200 μmol, 100 μmol, 90 μmol, 80 μmol, 70 μmol, 60 μmol, 50 μmol, 40 μmol, 30 μmol, 20 μmol, 10 μmol, 9 μmol, 8 mol, 7 μmol, 6 μmol, 5 μmol, 4 μmol, 3 μmol, 2 μmol, 1 μmol, 900 nmol, 800 nmol, 700 nmol, 600 nmol, 500 nmol, 400 nmol, 300 nmol, 200 nmol, 100 nmol, 90 nmol, 80 nmol, 70 nmol, 60 nmol, 50 nmol, 40 nmol, 30 nmol, 20 nmol, 10 nmol, 9 nmol, 8 nmol, 7 nmol, 6 nmol, 5 nmol, 4 nmol, 3 nmol, 2 nmol, 1 nmol, 900 pmol, 800 pmol, 700 pmol, 600 pmol, 500 pmol, 400 pmol, 300 pmol, 200 pmol, 100 pmol, 90 pmol, 80 pmol, 70 pmol, 60 pmol, 50 pmol, 40 pmol, 30 pmol, 20 pmol, 10 pmol, 9 pmol, 8 pmol, 7 pmol, 6 pmol, 5 pmol, 4 pmol, 3 pmol, 2 pmol, 1 pmol, 900 fmol, 800 fmol, 700 fmol, 600 fmol, 500 fmol, 400 fmol, 300 fmol, 200 fmol, 100 fmol, 90 fmol, 80 fmol, 70 fmol, 60 fmol, 50 fmol, 40 fmol, 30 fmol, 20 fmol, 10 fmol, 9 fmol, 8 fmol, 7 fmol, 6 fmol, 5 fmol, 4 fmol, 3 fmol, 2 fmol, 1 fmol, or less of target molecules. The plurality of target molecules may comprise a number of target molecules that is within a range defined by any two of the preceding values.

The plurality of target molecules may comprise a concentration of at least about 1 nanomolar (nM), 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 micromolar (μM), 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 millimolar (mM), 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, or more. The plurality of target molecules may comprise a concentration of at most about 100 mM, 90 mM, 80 mM, 70 mM, 60 mM, 50 mM, 40 mM, 30 mM, 20 mM, 10 mM, 9 mM, 8 mM, 7 mM, 6 mM, 5 mM, 4 mM, 3 mM, 2 mM, 1 mM, 900 μM, 800 μM, 700 μM, 600 μM, 500 μM, 400 μM, 300 μM, 200 μM, 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less. The plurality of target molecules may comprise a concentration that is within a range defined by any two of the preceding values. For instance, the plurality of target molecules may comprise a concentration between about 1 μM and about 10 mM.

In various embodiments, the first location comprises a first surface. For instance, the first surface may comprise a glass surface. The glass surface may be functionalized with a sensing region. For instance, the glass surface may comprise a thin film of a noble metal, such as gold, silver, platinum, or palladium, or any combination or alloy thereof, for use in SPR detection. The first surface may be functionalized with a surface functionalization moiety, such as an amine, carboxyl, or other moiety to permit attachment of a plurality of probe molecules to the first surface.

In various embodiments, the first surface may comprise a dielectric substrate that is subject to optical interrogation. The dielectric substrate may be functionalized with a barrier layer encompassing a sensing region that is optically interrogated. For instance, in the case of surface plasmon resonance-based optical interrogation, the glass surface may comprise a thin film of a noble metal, such as gold coated with a chemisorbed self-assembled monolayer of alkanethiol forming a suitable barrier layer. The barrier layer may have a thickness of at least about 1 nm, 2 nm, 3 nm, 4 nm, 5 nm, 6 nm, 7 nm, 8 nm, 9 nm, 10 nm, or more.

Metallic coatings may not be required in optical interferometry, ellipsometry, and diffraction-grating based optical biosensors. For measurements using these techniques, a suitable barrier layer may be produced by self-assembly directly onto the dielectric substrate using silanization reactions. A matrix may then be grafted to the surface using any number of covalent linkage chemistries. Epoxide reactions, halogen-based reactions, and carbidimide reactions may be utilized to allow hydroxyl groups, carboxyl groups, thiol groups, or amine groups of the barrier layer or matrix to be cross-linked irreversibly. The plurality of probe molecules may be covalently attached to the barrier layer or the hydrogel using similar cross-linkage chemistries.

In various embodiments, the first location comprises a matrix bound to the first surface. The matrix may comprise a hydrogel. The hydrogel may comprise a network of crosslinked polymer chains, which may be hydrophilic. The hydrogel may be formed as a colloidal gel with water as a dispersion medium. The hydrogel may be held together by cross-linking between the hydrophilic polymer chains. The hydrogel may comprise at least about 50%, 55%, 60%, 65%, 70%, 75%, 80%, 85%, 90%, 95%, or more of water by weight. The hydrogel may comprise at most about 95%, 90%, 85%, 80%, 75%, 70%, 65%, 60%, 55%, 50%, or less of water by weight. The hydrogel may comprise an amount of water than is within a range defined by any two of the preceding values. The hydrogel may comprise polymer chains formed from a variety of monomers, such as polyvinyl alcohol, polyethylene glycol, sodium polyacrylate, acrylate polymers, acrylate copolymers, collage, gelatin, fibrin, or any other suitable polymer chains. The hydrogel may be composed of substantially non-cross-linked, unbranched, polysaccharide chains such as dextran, alginate, karaya possessing a fraction of carboxylic acid functional groups, or other electronegative functional groups that support matric expansion and functionalization.

At least a portion of the matrix may comprise a matrix functionalization moiety. The matrix functionalization moiety may comprise an amine, carboxyl, or other moiety to permit attachment of a plurality of probe molecules to the matrix. The portion of the matrix comprising the matrix functionalization moiety may be at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, of the matrix. The portion of the matrix comprising the matrix functionalization moiety may be at most about 95%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less, of the matrix. The portion of the matrix comprising the matrix functionalization moiety may be within a range defined by any two of the preceding values.

In various embodiments, the matrix comprises a thickness of at least about 1 nanometer (nm), 2 nm, 3 nm, 4 nm, 5 nm, 6 nm, 7 nm, 8 nm, 9 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1,000 nm, or more. The matrix may comprise a thickness of at most about 1,000 nm, 900 nm, 800 nm, 700 nm, 600 nm, 500 nm, 400 nm, 300 nm, 200 nm, 100 nm, 90 nm, 80 nm, 70 nm, 60 nm, 50 nm, 40 nm, 30 nm, 20 nm, 10 nm, 9 nm, 8 nm, 7 nm, 6 nm, 5 nm, 4 nm, 3 nm, 2 nm, 1 nm, or less. The matrix may comprise a thickness that is within a range defined by any two of the preceding values.

In various embodiments, the first location comprises a plurality of probe molecules. The plurality of probe molecules may be similar to the probe molecules P described herein with respect to FIGS. 1 and 2. The plurality of probe molecules may comprise any number of probe molecules. For instance, the plurality of probe molecules may comprise any number of molecules described herein with respect to the number of target molecules.

In various embodiments, the plurality of probe molecules is bound to the hydrogel via a second linker. For instance, the plurality of probe molecules may be bound to the hydrogel by a linker having a binding moiety that is complementary to a hydrogel functionalization moiety described herein.

In various embodiments, the method 300 comprises a second step 320 of monitoring, via a detection method, a first amount of an intermolecular complex formed between the plurality of probe molecules and the plurality of target molecules. The intermolecular complex may be similar to the first intermolecular complex BP described herein with respect to FIGS. 1 and 2. The detection method used to perform the monitoring may comprise an SPR method, as described herein. Alternatively, the detection method used to perform the monitoring may comprise a variety of optical and/or non-optical methods, such as an interferometry method, an optical interferometry method, a biolayer interferometry method, an ellipsometry method, a waveguide-based sensing method, a photonic crystal method, a photonically excited acoustic resonator method, a fluorescence based method, a total internal reflection fluorescence based method, an acoustic resonator method, a quartz crystal resonator method, an electrical sensing method, a graphene based electrical sensing method, or any other detection method that may occur to one having skill in the art.

In various embodiments, the method 300 comprises a third step 330 of, when the first amount exceeds a threshold value, stopping the introduction of the plurality of target molecules and introducing a plurality of competitive inhibitor molecules to the first location. The plurality of competitive inhibitor molecules may be similar to the competitive inhibitor molecules A described herein with respect to FIGS. 1 and 2. The plurality of competitive inhibitor molecules may comprise any number of competitive inhibitor molecules. For instance, the plurality of competitive inhibitor molecules may comprise any number of molecules described herein with respect to the number of target molecules.

In various embodiments, the method 300 comprises a fourth step 340 of monitoring, via the detection method, a second amount of the intermolecular complex. The second amount may correspond to the amount of the intermolecular complex remaining within the matrix. The second amount of the intermolecular complex may be monitored at time intervals in order to generate a time-dependent dissociation curve. The curve may be acquired at a data acquisition rate of at least about 0.001 Hertz (Hz), 0.002 Hz, 0.003 Hz, 0.004 Hz, 0.005 Hz, 0.006 Hz, 0.007 Hz, 0.008 Hz, 0.009 Hz, 0.01 Hz, 0.02 Hz, 0.03 Hz, 0.04 Hz, 0.05 Hz, 0.06 Hz, 0.07 Hz, 0.08 Hz, 0.09 Hz, 0.1 Hz, 0.2 Hz, 0.3 Hz, 0.4 Hz, 0.5 Hz, 0.6 Hz, 0.7 Hz, 0.8 Hz, 0.9 Hz, 1 Hz, 2 Hz, 3 Hz, 4 Hz, 5 Hz, 6 Hz, 7 Hz, 8 Hz, 9 Hz, 10 Hz, 20 Hz, 30 Hz, 40 Hz, 50 Hz, 60 Hz, 70 Hz, 80 Hz, 90 Hz, 100 Hz, 200 Hz, 300 Hz, 400 Hz, 500 Hz, 600 Hz, 700 Hz, 800 Hz, 900 Hz, 1,000 Hz, or more. The curve may be acquired at a data acquisition rate of at most about 1,000 Hz, 900 Hz, 800 Hz, 700 Hz, 600 Hz, 500 Hz, 400 Hz, 300 Hz, 200 Hz, 100 Hz, 90 Hz, 80 Hz, 70 Hz, 60 Hz, 50 Hz, 40 Hz, 30 Hz, 20 Hz, 10 Hz, 9 Hz, 8 Hz, 7 Hz, 6 Hz, 5 Hz, 4 Hz, 3 Hz, 2 Hz, 1 Hz, 0.9 Hz, 0.8 Hz, 0.7 Hz, 0.6 Hz, 0.5 Hz, 0.4 Hz, 0.3 Hz, 0.2 Hz, 0.1 Hz, 0.09 Hz, 0.08 Hz, 0.07 Hz, 0.06 Hz, 0.05 Hz, 0.04 Hz, 0.03 Hz, 0.02 Hz, 0.01 Hz, 0.009 Hz, 0.008 Hz, 0.007 Hz, 0.006 Hz, 0.005 Hz, 0.004 Hz, 0.003 Hz, 0.002 Hz, 0.001 Hz, or less. The curve may be acquired at a data acquisition rate that is within a range defined by any two of the preceding values.

In various embodiments, the method 300 comprises a fifth step 350 of determining the kinetic parameter based upon the second amount. The kinetic parameter may be determined based upon the second amount in accordance with Equations (6)-(11), (12)-(20), and/or (21)-(31) described herein with respect to FIGS. 1 and 2. The kinetic parameter may comprise an association rate constant (for instance, kₐ or kₐ′ as described herein with respect to FIGS. 1 and 2) between the plurality of target molecules and the plurality of competitive inhibitor molecules. The kinetic parameter may comprise a dissociation rate constant (for instance, k_{d} or k_{d}′ as described herein with respect to FIGS. 1 and 2) between the plurality of target molecules and the plurality of competitive inhibitor molecules. The kinetic parameter may be at least about 1 molar⁻¹ seconds⁻¹ (M⁻¹s⁻¹), 2 M⁻¹s⁻¹, 3 M⁻¹s⁻¹, 4 M⁻¹s⁻¹, 5 M⁻¹s⁻¹, 6 M⁻¹s⁻¹, M⁻¹s⁻¹, 8 M⁻¹s⁻¹, 9 M⁻¹s⁻¹, 10 M⁻¹s⁻¹, 20 M⁻¹s⁻¹, 30 M⁻¹s⁻¹, 40 M⁻¹s⁻¹, 50 M⁻¹s⁻¹, 60 M⁻¹s⁻¹, 70 M⁻¹s⁻¹, 80 M⁻¹s⁻¹, 90 M⁻¹s⁻¹, 100 M⁻¹s⁻¹, 200 M⁻¹s⁻¹, 300 M⁻¹s⁻¹, 400 M⁻¹s⁻¹, 500 M⁻¹s⁻¹, 600 M⁻¹s⁻¹, 700 M⁻¹s⁻¹, 800 M⁻¹s⁻¹, 900 M⁻¹s⁻¹, 1,000 M⁻¹s⁻¹, 2,000 M⁻¹s⁻¹, 3,000 M⁻¹s⁻¹, 4,000 M⁻¹s⁻¹, 5,000 M⁻¹s⁻¹, 6,000 M⁻¹s⁻¹, 7,000 M⁻¹s⁻¹, 8,000 M⁻¹s⁻¹, 9,000 M⁻¹s⁻¹, 10,000 M⁻¹s⁻¹, 20,000 M⁻¹s⁻¹, 30,000 M⁻¹s⁻¹, 40,000 M⁻¹s⁻¹, 50,000 M⁻¹s⁻¹, 60,000 M⁻¹s⁻¹, 70,000 M⁻¹s⁻¹, 80,000 M⁻¹s⁻¹, 90,000 M⁻¹s⁻¹, 100,000 M⁻¹s⁻¹, 200,000 M⁻¹s⁻¹, 300,000 M⁻¹s⁻¹, 400,000 M⁻¹s⁻¹, 500,000 M⁻¹s⁻¹, 600,000 M⁻¹s⁻¹, 700,000 M⁻¹s⁻¹, 800,000 M⁻¹s⁻¹, 900,000 M⁻¹s⁻¹, 1,000,000 M⁻¹s⁻¹, 2,000,000 M⁻¹s⁻¹, 3,000,000 M⁻¹s⁻¹, 4,000,000 M⁻¹s⁻¹, 5,000,000 M⁻¹s⁻¹, 6,000,000 M⁻¹s⁻¹, 7,000,000 M⁻¹s⁻¹, 8,000,000 M⁻¹s⁻¹, 9,000,000 M⁻¹s⁻¹, 10,000,000 M⁻¹s⁻¹, 20,000,000 M⁻¹s⁻¹, 30,000,000 M⁻¹s⁻¹, 40,000,000 M⁻¹s⁻¹, 50,000,000, M⁻¹s⁻¹, 60,000,000 M⁻¹s⁻¹, 70,000,000 M⁻¹s⁻¹, 80,000,000 M⁻¹s⁻¹, 90,000,000 M⁻¹s⁻¹, 100,000,000 M⁻¹s⁻¹, 200,000,000 M⁻¹s⁻¹, 300,000,000 M⁻¹s⁻¹, 400,000,000 M⁻¹s⁻¹, 500,000,000 M⁻¹s⁻¹, 600,000,000 M⁻¹s⁻¹, 700,000,000 M⁻¹s⁻¹, 800,000,000 M⁻¹s⁻¹, 900,000,000 M⁻¹s⁻¹, 1,000,000,000 M⁻¹s⁻¹, or more. The kinetic parameter may be at most about 1,000,000,000 M⁻¹s⁻¹, 900,000,000 M⁻¹s⁻¹, 800,000,000 M⁻¹s⁻¹, 700,000,000 M⁻¹s⁻¹, 600,000,000 M⁻¹s⁻¹, 500,000,000 M⁻¹s⁻¹, 400,000,000 M⁻¹s⁻¹, 300,000,000 M⁻¹s⁻¹, 200,000,000 M⁻¹s⁻¹, 100,000,000 M⁻¹s⁻¹, 90,000,000 M⁻¹s⁻¹, 80,000,000 M⁻¹s⁻¹, 70,000,000 M⁻¹s⁻¹, 60,000,000 M⁻¹s⁻¹, 50,000,000 M⁻¹s⁻¹, 40,000,000 M⁻¹s⁻¹, 30,000,000 M⁻¹s⁻¹, 20,000,000 M⁻¹s⁻¹, 10,000,000 M⁻¹s⁻¹, 9,000,000 M⁻¹s⁻¹, 8,000,000 M⁻¹s⁻¹, 7,000,000 M⁻¹s⁻¹, 6,000,000 M⁻¹s⁻¹, 5,000,000 M⁻¹s⁻¹, 4,000,000 M⁻¹s⁻¹, 3,000,000 M⁻¹s⁻¹, 2,000,000 M⁻¹s⁻¹, 1,000,000 M⁻¹s⁻¹, 900,000 M⁻¹s⁻¹, 800,000 M⁻¹s⁻¹, 700,000 M⁻¹s⁻¹, 600,000 M⁻¹s⁻¹, 500,000 M⁻¹s⁻¹, 400,000 M⁻¹s⁻¹, 300,000 M⁻¹s⁻¹, 200,000 M⁻¹s⁻¹, 100,000 M⁻¹s⁻¹, 90,000 M⁻¹s⁻¹, 80,000 M⁻¹s⁻¹, 70,000 M⁻¹s⁻¹, 60,000 M⁻¹s⁻¹, 50,000 M⁻¹s⁻¹, 40,000 M⁻¹s⁻¹, 30,000 M⁻¹s⁻¹, 20,000 M⁻¹s⁻¹, 10,000 M⁻¹s⁻¹, 9,000 M⁻¹s⁻¹, 8,000 M⁻¹s⁻¹, 7,000 M⁻¹s⁻¹, 6,000 M⁻¹s⁻¹, 5,000 M⁻¹s⁻¹, 4,000 M⁻¹s⁻¹, 3,000 M⁻¹s⁻¹, 2,000 M⁻¹s⁻¹, 1,000 M⁻¹s⁻¹, 900 M⁻¹s⁻¹, 800 M⁻¹s⁻¹, 700 M⁻¹s⁻¹, 600 M⁻¹s⁻¹, 500 M⁻¹s⁻¹, 400 M⁻¹s⁻¹, 300 M⁻¹s⁻¹, 200 M⁻¹s⁻¹, 100 M⁻¹s⁻¹, 90 M⁻¹s⁻¹, 80 M⁻¹s⁻¹, 70 M⁻¹s⁻¹, 60 M⁻¹s⁻¹, 50 M⁻¹s⁻¹, 40 M⁻¹s⁻¹, 30 M⁻¹s⁻¹, 20 M⁻¹s⁻¹, 10 M⁻¹s⁻¹, 9 M⁻¹s⁻¹, 8 M⁻¹s⁻¹, 7 M⁻¹s⁻¹, 6 M⁻¹s⁻¹, 5 M⁻¹s⁻¹, 4 M⁻¹s⁻¹, 3 M⁻¹s⁻¹, 2 M⁻¹s⁻¹, 1 M⁻¹s⁻¹, or less. The kinetic parameter may be within a range defined by any two of the preceding values.

The kinetic parameter may be a transient kinetic parameter (i.e., a kinetic parameter associated with transient kinetics). The transient kinetic parameter may be at least about 10,000,000 M⁻¹s⁻¹, 20,000,000 M⁻¹s⁻¹, 30,000,000 M⁻¹s⁻¹, 40,000,000 M⁻¹s⁻¹, 50,000,000, M⁻¹s⁻¹, 60,000,000 M⁻¹s⁻¹, 70,000,000 M⁻¹s⁻¹, 80,000,000 M⁻¹s⁻¹, 90,000,000 M⁻¹s⁻¹, 100,000,000 M⁻¹s⁻¹, 200,000,000 M⁻¹s⁻¹, 300,000,000 M⁻¹s⁻¹, 400,000,000 M⁻¹s⁻¹, 500,000,000 M⁻¹s⁻¹, 600,000,000 M⁻¹s⁻¹, 700,000,000 M⁻¹s⁻¹, 800,000,000 M⁻¹s⁻¹, 900,000,000 M⁻¹s⁻¹, 1,000,000,000 M⁻¹s⁻¹, or more. The transient kinetic parameter may be at most about 1,000,000,000 M⁻¹s⁻¹, 900,000,000 M⁻¹s⁻¹, 800,000,000 M⁻¹s⁻¹, 700,000,000 M⁻¹s⁻¹, 600,000,000 M⁻¹s⁻¹, 500,000,000 M⁻¹s⁻¹, 400,000,000 M⁻¹s⁻¹, 300,000,000 M⁻¹s⁻¹, 200,000,000 M⁻¹s⁻¹, 100,000,000 M⁻¹s⁻¹, 90,000,000 M⁻¹s⁻¹, 80,000,000 M⁻¹s⁻¹, 70,000,000 M⁻¹s⁻¹, 60,000,000 M⁻¹s⁻¹, 50,000,000 M⁻¹s⁻¹, 40,000,000 M⁻¹s⁻¹, 30,000,000 M⁻¹s⁻¹, 20,000,000 M⁻¹s⁻¹, 10,000,000 M⁻¹s⁻¹, or less. The transient kinetic parameter may be within range defined by any two of the preceding values. For instance, the transient kinetic parameter may be between about 10,000,000 M⁻¹s⁻¹ and about 100,000,000 M⁻¹s⁻¹, between about 10,000,000 M⁻¹s⁻¹ and about 1,000,000,000 M⁻¹s⁻¹, or between about 100,000,000 M⁻¹s⁻¹ and about 1,000,000,000 M⁻¹s⁻¹.

In various embodiments, the kinetic parameter is within a predetermined range. The method 300 may be sensitized to one or more parameters associated with the matrix or the first surface, as described herein with respect to FIGS. 1 and 2. For instance, one or more dimensions of the matrix may be chosen to sensitize the method to the predetermined range. For instance, a thickness of the matrix may be chosen to sensitize the method to the predetermined range. Any matrix thickness described herein may be chosen. A density or extent of crosslinking of the matrix may be chosen to sensitize the method to the predetermined range. A density of the plurality of probe molecules (in the matrix or on the first surface) may be chosen to sensitize the method to the predetermined range.

In various embodiments, the method 300 is implemented using a channel-based system, as described herein with respect to FIG. 5. In such embodiments, the first step 310 may comprise starting a flow of the plurality of target molecules through the first channel and the third step 330 may comprise, when the first amount exceeds a threshold value, stopping the flow of the plurality of target molecules through the first channel and starting a flow of the plurality of competitive inhibitor molecules through the first channel. Optionally these serial flows may be interrupted by continuous flow of a buffer that washes the flow cell conduits. In such embodiments, the method may further comprise monitoring, via the detection method, a second location. The second location may comprise a second channel in the flow cell. The second location may comprise a second surface. The second surface may or may not comprise a probe molecule bound to the second surface. The second surface may or may not comprise a matrix. Monitoring the second location may provide a reference signal that may be compared to the signal associated with the first and second amounts of the intermolecular complex, enhancing the SNR. The second location may be located at least about 1 nm, 2 nm, 3 nm, 4 nm, 5 nm, 6 nm, 7 nm, 8 nm, 9 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, or more from the first location. The second location may be located at most about 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, 9 μm, 8 μm, 7 μm, 6 μm, 5 μm, 4 μm, 3 μm, 2 μm, 1 μm, 900 nm, 800 nm, 700 nm, 600 nm, 500 nm, 400 nm, 300 nm, 200 nm, 100 nm, 90 nm, 80 nm, 70 nm, 60 nm, 50 nm, 40 nm, 30 nm, 20 nm, 10 nm, 9 nm, 8 nm, 7 nm, 6 nm, 5 nm, 4 nm, 3 nm, 2 nm, 1 nm, or less from the first location. The second location may be located a distance from the first location that is within a range defined by any two of the preceding values.

In various embodiments, the method 300 is implemented using a well-based system, as described herein with respect to FIG. 6. In such embodiments, the first step 310 may comprise placing (for instance, by pipetting, auto-pipetting, injecting, or auto-injecting) the plurality of target molecules into the first well and the third step 330 may comprise, when the first amount exceeds a threshold value, stopping the placement of the plurality of target molecules into the first well and placing the plurality of competitive inhibitor molecules into the first well. In such embodiments, the method may further comprise monitoring, via the detection method, a second location. The second location may comprise a second well in the well plate. The second location may comprise a second surface. The second surface may or may not comprise a probe molecule bound to the second surface. The second surface may or may not comprise a matrix. Monitoring the second location may provide a reference signal that may be compared to the signal associated with the first and second amounts of the intermolecular complex, enhancing the SNR. The second location may be located any distance from the first location described herein.

In various embodiments, step 320 comprises, for a plurality of points in time: detecting, via the detection method, a detection signal indicative of the first amount of intermolecular complex and step 330 comprises, for a plurality of points in time, (i) comparing the first amount with the threshold value; and (ii) if the first amount exceeds the threshold value, stopping the introduction of the plurality of target molecules and introducing the plurality of competitive inhibitor molecules to the first location. The first amount may increase over the plurality of points in time, as described herein with respect to FIGS. 1 and 2.

In various embodiments, step 340 comprises, for a plurality of points in time: detecting, via the detection method, a detection signal indicative of the second amount of the intermolecular complex. The second amount may decrease over the plurality of points in time, as described herein with respect to FIGS. 1 and 2.

In various embodiments, the method 300 further comprises, prior to step 310, binding the matrix to the first surface and binding the plurality of probe molecules to the matrix.

In various embodiments, the method 300 may be implemented without the need to monitor the first amount of the intermolecular complex. For instance, the method 300 may be implemented by estimating an amount of time required for the first amount of the intermolecular complex to reach the threshold level. Such an estimate may be determined by prior knowledge or prior guesses as to the values of kinetic parameters related to the formation of the intermolecular complex. Thus, the method 300 may be modified to eliminate step 320.

### Detection Via Surface Plasmon Resonance (SPR)

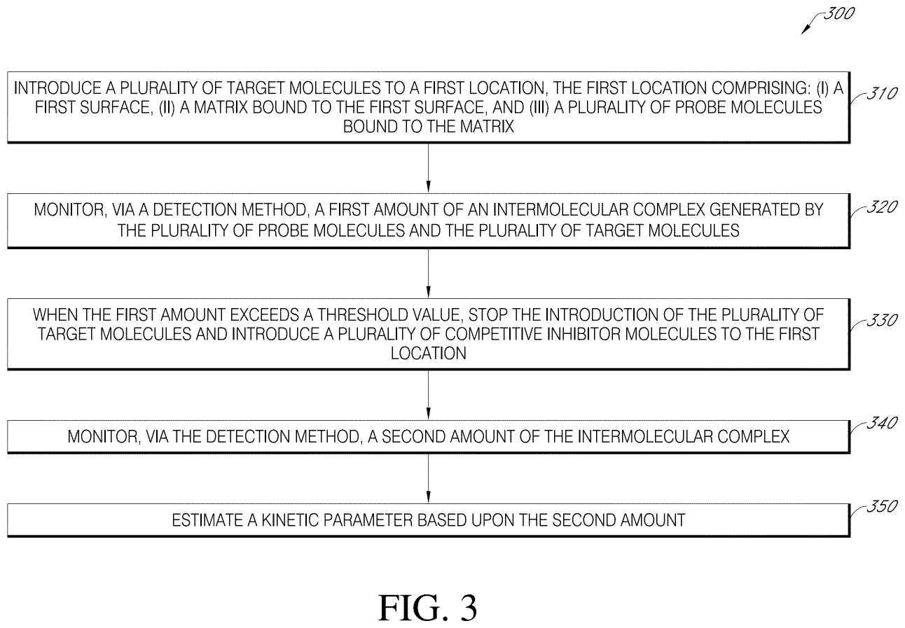

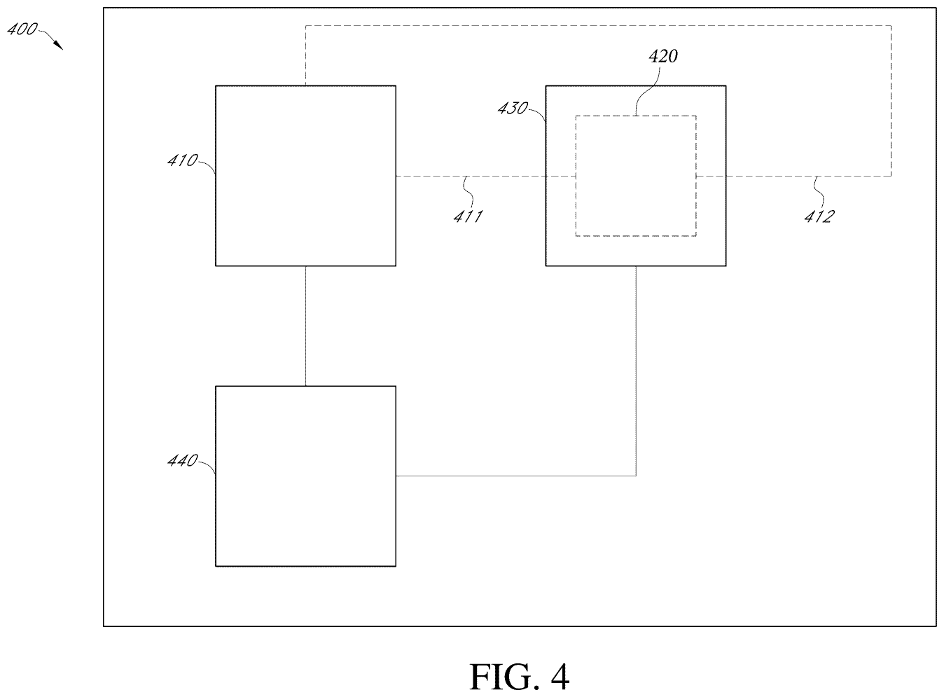

In various embodiments, the methods and systems described herein (for instance, method 300 described herein with respect to FIG. 3 or system 400 described herein with respect to FIG. 4) are implemented using SPR detection. The SPR detection may be monochromatic SPR detection or multi-wavelength SPR detector. During a monochromatic SPR experiment, substantially monochromatic light (for instance, from a narrow bandwidth light-emitting diode or laser) may be focused and directed to a thin metallic film (such as a film comprising gold, silver, platinum, or palladium, or any combination or alloy thereof). An optical detector may detect the light reflected from the thin metallic film.

In various embodiments, the thin metallic film is adhered to one side of a substrate. The matrix and probe molecules described herein may be located on the opposite side of the substrate from the thin metallic film. As the SPR experiment progresses, the angle of incidence of the laser light may be scanned over a range of angles. At some critical angle, a substantial fraction of the energy associated with the laser light may be lost to the metallic surface as evanescent plasmon waves. This critical angle may be determined by noting the angle of incidence at which the reflected light detected by the detector falls to its lowest value.

In various embodiments, the critical angle is dependent upon the index of refraction on the opposite side of the substrate, which may itself depend upon the amount of matrix, probe molecules, target molecules bound to probe molecules, target molecules bound to competitive inhibitor molecules, or solution present on the opposite side of the substrate. In the methods and systems described herein, the amount of matrix, probe molecules, and solution present on the opposite side of the substrate may generally be relatively fixed. As such, the index of refraction may be strongly correlated with the amount of target molecules bound to probe molecules or target molecules bound to competitive inhibitor molecules.

In various embodiments, as the target molecules bind to and unbind from the probe molecules or the competitive inhibitor molecules, the refractive index (and therefore the critical angle) changes. The angle of incidence may be scanned to determine how the critical angle (and therefore the refractive index) changes during the course of the SPR experiment. This information may be presented to an experimenter as a binding response curve, which may be similar in form to the binding response curves shown in FIG. 2.

During a multi-wavelength SPR experiment, multiple wavelengths of light may be focused and directed to the thin metallic film. An optical detector may detect one or more wavelengths of light reflected from the thin metallic film. Changes in the wavelengths detected by the optical detector may be indicative of a change in the amount of target molecules bound to probe molecules or target molecules bound to competitive inhibitor molecules.

In various embodiments, the methods and systems described herein (for instance, method 300 described herein with respect to FIG. 3 or system 400 described herein with respect to FIG. 4) are implemented using non-SPR detection techniques. For instance, the methods and systems may be implemented using optical biosensors based on planar waveguides, fiber optics, photonic crystals, diffraction gratings, and the like. Optical techniques such as optical interferometry or ellipsometry may be employed, as described herein.

### Systems for Determining a Kinetic Parameter

FIG. 4 is a block diagram of a system 400 for determining a kinetic parameter. The system 400 may be configured to implement the method 300 described herein. In various embodiments, the system comprises a flow manifold 410, an assay module 420, a detector 430, and a controller 440. Dashed lines in FIG. 4 indicate that an element is located behind or underneath another element. The flow manifold may be capable of fluidically coupling to the assay module. The flow manifold may be fluidically coupled to the assay module by at least one fluid supply line 411 and at least one fluid return line 412. Although depicted as being fluidically coupled to the assay module by a single fluid supply line and a single fluid return line in FIG. 4, the flow manifold may be fluidically coupled to the assay module by any number of fluid supply lines and any number of fluid return lines. For instance, the flow manifold may be fluidically coupled to the assay module by at least about 1, 2, 3, 4, 5, 6, 7, 8, 9, 10, 20, 30, 40, 50, 60, 70, 80, 90, 100, 200, 300, 400, 500, 600, 700, 800, 900, 1,000, or more fluid supply lines or fluid return lines. The flow manifold may be fluidically coupled to the assay module by at most about 1,000, 900, 800, 700, 600, 500, 400, 300, 200, 100, 90, 80, 70, 60, 50, 40, 30, 20, 10, 9, 8, 7, 6, 5, 4, 3, 2, 1 or fluid supply lines or fluid return lines. The assay module may comprise a number of fluid supply lines or fluid return lines that is within a range defined by any two of the preceding values. The assay module 420 may comprise a channel-based assay module 500 or a well-based assay module 600 described herein.

FIG. 5 is a block diagram of a channel-based assay module 500 for determining a kinetic parameter. In various embodiments, the assay module comprises a first location comprising a first channel 510. The first channel may comprise a first inlet 511 and a first outlet 512. The first inlet may be fluidically coupled to the flow manifold by a fluid supply line. The first outlet may be fluidically coupled to the flow manifold by a fluid return line. The first channel may comprise a first surface 700, a matrix 710 bound to the first surface, and a plurality of probe molecules 720, each as described herein with respect to FIG. 7.

FIG. 6 is a block diagram of a well-based assay module 600 for determining a kinetic parameter. In various embodiments, the assay module comprises a first location comprising a first well 610. The first well may be fluidically coupled to the flow manifold by a fluid supply line. The fluid supply line may be coupled to a probe, pipette, or autosampler. The first well may be fluidically coupled to the flow manifold by a fluid return line. The first well may comprise a first surface 700, a matrix 710 bound to the first surface, and a plurality of probe molecules 720, each as described herein with respect to FIG. 7.

FIG. 7 is a block diagram of a functionalized surface 700. In various embodiments, the surface comprises a glass surface. All or a portion of the surface may be functionalized with a sensing region 701, such as a thin film of any noble metal described herein. A matrix 710 may be bound to the surface. The matrix may comprise any matrix described herein, such as any hydrogel described herein. A plurality of probe molecules 720 may be embedded within the matrix. The plurality of probe molecules may be bound to the surface or the matrix. For instance, the plurality of probe molecules may be bound to the matrix via a linker 721. As shown in FIG. 7, a portion of the plurality of probe molecules may be bound to the surface.

Returning to the discussion of FIG. 5, in various embodiments, the assay module 500 further comprises a second channel 520. The second channel may comprise a second inlet 521 and a second outlet 522. Although depicted as separate in FIG. 5, the first outlet and second outlet may be superimposable and of any shape profile defined by two inlets and a single outlet. The second inlet may be fluidically coupled to the flow manifold by a fluid supply line. The second outlet may be fluidically coupled to the flow manifold by a fluid return line. The second channel may comprise a second surface 523. The second surface may be functionalized with a sensing region 524, such as a thin film of any noble metal described herein. The second surface may operate as a reference surface for obtaining a background signal, such as a background SPR signal. Use of such a reference surface may allow increased SNR in signals obtained from the first surface, as described herein. As such, the second surface may not comprise a probe molecule bound thereto or may not comprise a matrix bound thereto, as described herein.

Although depicted as comprising two channels, two inlets, and two outlets, and coupled to two fluid supply lines and two fluid return lines in FIG. 5, the assay module 500 may comprise any number of channels, inlets, or outlets, and may be coupled to any number of fluid supply lines and fluid return lines. For instance, the assay module may comprise at least about 1, 2, 3, 4, 5, 6, 7, 8, 9, 10, 20, 30, 40, 50, 60, 70, 80, 90, 100, 200, 300, 400, 500, 600, 700, 800, 900, 1,000, or more channels, inlets, or outlets, and may be coupled to at least about 1, 2, 3, 4, 5, 6, 7, 8, 9, 10, 20, 30, 40, 50, 60, 70, 80, 90, 100, 200, 300, 400, 500, 600, 700, 800, 900, 1,000, or more fluid supply lines or fluid return lines. The assay module may comprise at most about 1,000, 900, 800, 700, 600, 500, 400, 300, 200, 100, 90, 80, 70, 60, 50, 40, 30, 20, 10, 9, 8, 7, 6, 5, 4, 3, 2, or 1 channels, inlets, or outlets, and may be coupled to at most about 1,000, 900, 800, 700, 600, 500, 400, 300, 200, 100, 90, 80, 70, 60, 50, 40, 30, 20, 10, 9, 8, 7, 6, 5, 4, 3, 2, or 1 fluid supply lines or fluid return lines. The assay module may comprise a number of channels, inlets, or outlets that is within a range defined by any two of the preceding values, and may be coupled to a number of fluid supply lines or fluid return lines that is within a range defined by any two of the preceding values. In some cases, a single location may be located at the intersection of a plurality of channels (each comprising an inlet and an outlet and coupled to the flow manifold by a fluid supply line and a fluid return line). In such cases, each channel may be configured to supply different reactants (such as target molecules and competitive inhibitor molecules described herein) to the location. In other cases, a single channel may be coupled to a plurality of fluid supply lines or a plurality of fluid return lines. In such cases, the different reactants may be delivered to the channel by different supply lines or fluid return lines.

Returning to the discussion of FIG. 6, in various embodiments, the assay module 600 further comprises a second well 620. The second well may be fluidically coupled to the flow manifold by a fluid supply line. The second well may be fluidically coupled to the flow manifold by a fluid return line. The second well may comprise a second surface 621. The second surface may be functionalized with a sensing region 622, such as a thin film of any noble metal described herein. The second surface may operate as a reference surface for obtaining a background signal, such as a background SPR signal. Use of such a reference surface may allow increased SNR in signals obtained from the first surface, as described herein. As such, the second surface may not comprise a probe molecule bound thereto or may not comprise a matrix bound thereto, as described herein.

Although depicted as comprising two wells, and coupled to two fluid supply lines and two fluid return lines in FIG. 6, the assay module 600 may comprise any number of wells, and may be coupled to any number of fluid supply lines and fluid return lines. For instance, the assay module may comprise at least about 1, 2, 3, 4, 5, 6, 7, 8, 9, 10, 20, 30, 40, 50, 60, 70, 80, 90, 100, 200, 300, 400, 500, 600, 700, 800, 900, 1,000, or more wells, and may be coupled to at least about 1, 2, 3, 4, 5, 6, 7, 8, 9, 10, 20, 30, 40, 50, 60, 70, 80, 90, 100, 200, 300, 400, 500, 600, 700, 800, 900, 1,000, or more fluid supply lines or fluid return lines. The assay module may comprise at most about 1,000, 900, 800, 700, 600, 500, 400, 300, 200, 100, 90, 80, 70, 60, 50, 40, 30, 20, 10, 9, 8, 7, 6, 5, 4, 3, 2, or 1 wells, and may be coupled to at most about 1,000, 900, 800, 700, 600, 500, 400, 300, 200, 100, 90, 80, 70, 60, 50, 40, 30, 20, 10, 9, 8, 7, 6, 5, 4, 3, 2, or 1 fluid supply lines or fluid return lines. The assay module may comprise a number of wells that is within a range defined by any two of the preceding values, and may be coupled to a number of fluid supply lines or fluid return lines that is within a range defined by any two of the preceding values. In some cases, a single well may be coupled to a plurality of fluid supply lines or a plurality of fluid return lines. In such cases, the different reactants may be delivered to the well by different supply lines or fluid return lines.

Returning to the discussion of FIG. 4, in various embodiments, the detector 430 comprises an optical detector. For instance, the detector may comprise a photodiode, photodiode array, avalanche photodiode, avalanche photodiode array, photomultiplier, photomultiplier array, photoresistor, photoresistor array, active-pixel sensor, complementary metal oxide semiconductor (CMOS) camera, charge-coupled device (CCD) camera, or any other optical detector. The detector may be capable of detecting an SPR signal, as described herein.

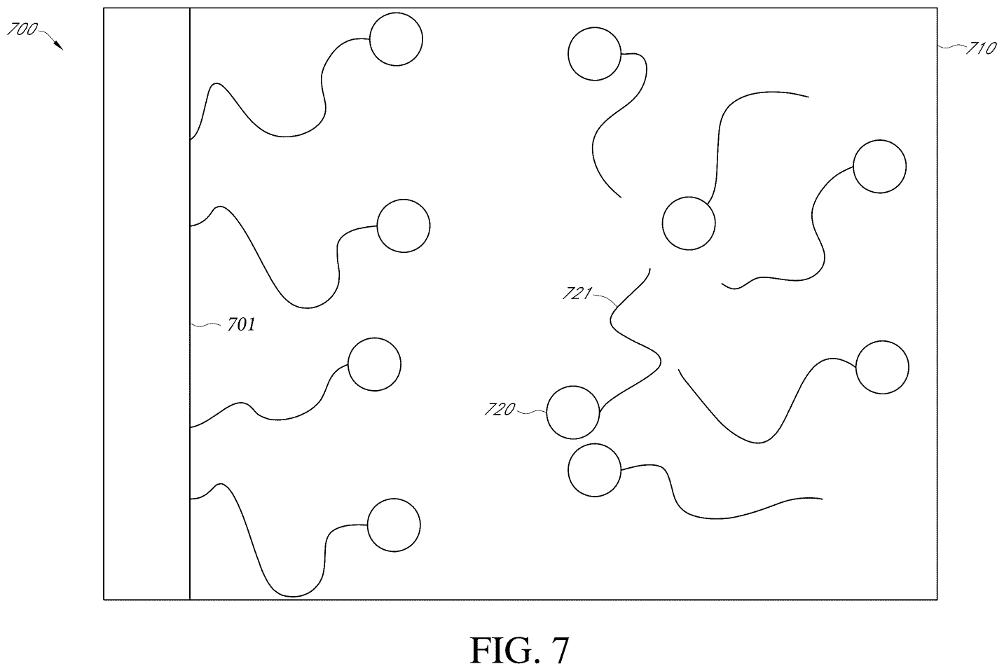

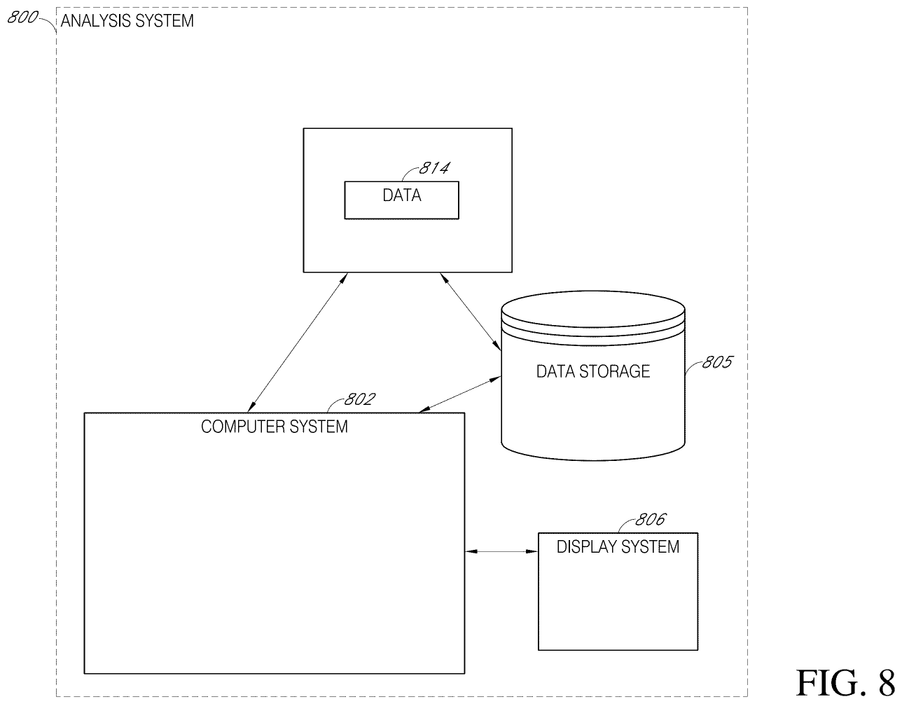

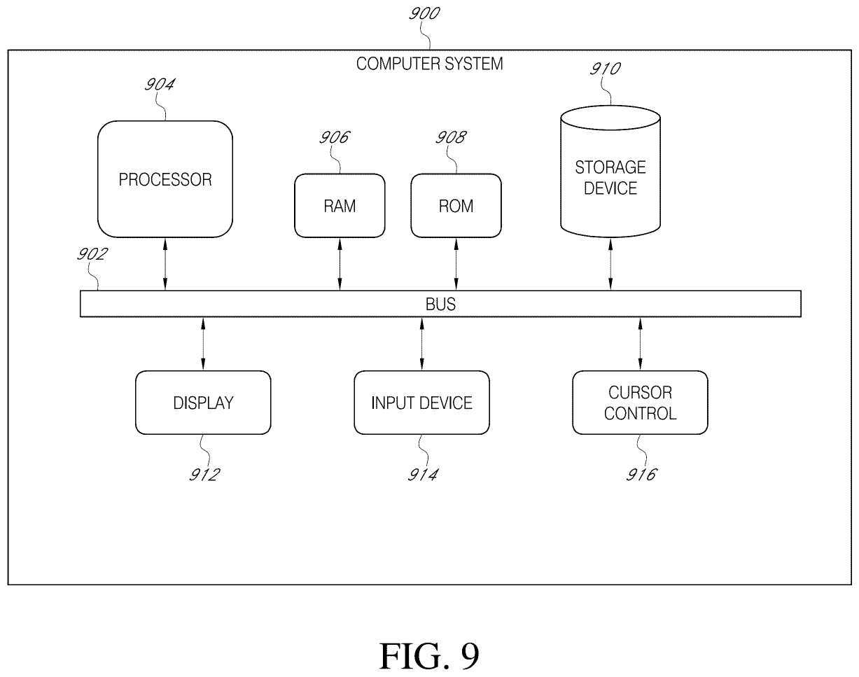

In various embodiments, the controller 440 is capable of directing the flow manifold or the detector to implement any or all of the steps 310, 320, 330, 340, and 350 of method 300 described herein. For instance, the controller may be capable of instructing or directing the flow manifold to introduce a first solution to the first location. The first solution may comprise any plurality of target molecules described herein. The controller may be capable of instructing or directing the detector to monitor a first amount of an intermolecular complex formed between the plurality of probe molecules and the plurality of target molecules. The controller may be capable of instructing or directing the flow manifold to stop the introduction of the first solution when the first amount exceeds a threshold value. The controller may be capable of directing the flow manifold to introduce a second solution to the first location when the first amount exceeds the threshold value. The second solution may comprise any plurality of competitive inhibitor molecules described herein. The controller may be configured to direct the detector to monitor a second amount of the intermolecular complex. The controller may be configured to determine the kinetic parameter based upon the second amount. The kinetic parameter may be determined based upon the second amount in accordance with Equations (6)-(11), (12)-(20), and/or (21)-(31) described herein with respect to FIGS. 1 and 2. The kinetic parameter may comprise any kinetic parameter described herein. The controller may comprise one or more elements of systems 800 or 900 described herein with respect to FIGS. 8 and 9, respectively.

### Computer-Implemented Systems for Determining a Kinetic Parameter

In various embodiments, at least a portion of the methods for estimating a kinetic parameter can be implemented via software, hardware, firmware, or a combination thereof.

That is, as depicted in FIG. 8, the methods and systems disclosed herein can be implemented on a computer-based system 800 for estimating a kinetic parameter. The system 800 may comprise a computer system such as computer system 802 (e.g., a computing device/analytics server). In various embodiments, the computer system 802 can be communicatively connected to a data storage 805 and a display system 806 via a direct connection or through a network connection (e.g., LAN, WAN, Internet, etc.). The computer system 802 can be configured to receive data, such as image feature data described herein. It should be appreciated that the computer system 802 depicted in FIG. 8 can comprise additional engines or components as needed by the particular application or system architecture.

FIG. 9 is a block diagram of a computer system in accordance with various embodiments. Computer system 900 may be an example of one implementation for computer system 802 described herein with respect to FIG. 8. In one or more examples, computer system 900 can include a bus 902 or other communication mechanism for communicating information, and a processor 904 coupled with bus 902 for processing information. In various embodiments, computer system 900 can also include a memory, which can be a random-access memory (RAM) 906 or other dynamic storage device, coupled to bus 902 for determining instructions to be executed by processor 904. Memory also can be used for storing temporary variables or other intermediate information during execution of instructions to be executed by processor 904. In various embodiments, computer system 900 can further include a read only memory (ROM) 908 or other static storage device coupled to bus 902 for storing static information and instructions for processor 904. A storage device 910, such as a magnetic disk or optical disk, can be provided and coupled to bus 402 for storing information and instructions.

In various embodiments, computer system 900 can be coupled via bus 902 to a display 912, such as a cathode ray tube (CRT) or liquid crystal display (LCD), for displaying information to a computer user. An input device 914, including alphanumeric and other keys, can be coupled to bus 902 for communicating information and command selections to processor 904. Another type of user input device is a cursor control 916, such as a mouse, a joystick, a trackball, a gesture input device, a gaze-based input device, or cursor direction keys for communicating direction information and command selections to processor 904 and for controlling cursor movement on display 912. This input device 914 typically has two degrees of freedom in two axes, a first axis (e.g., x) and a second axis (e.g., y), that allows the device to specify positions in a plane. However, it should be understood that input devices 912 allowing for three-dimensional (e.g., x, y and z) cursor movement are also contemplated herein.

Consistent with certain implementations of the present teachings, results can be provided by computer system 900 in response to processor 904 executing one or more sequences of one or more instructions contained in RAM 906. Such instructions can be read into RAM 906 from another computer-readable medium or computer-readable storage medium, such as storage device 910. Execution of the sequences of instructions contained in RAM 906 can cause processor 904 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 904 for execution. Such a medium can take many forms, including but not limited to, non-volatile media, volatile media, and transmission media. Examples of non-volatile media can include, but are not limited to, optical, solid state, magnetic disks, such as storage device 910. Examples of volatile media can include, but are not limited to, dynamic memory, such as RAM 906. Examples of transmission media can include, but are not limited to, coaxial cables, copper wire, and fiber optics, including the wires that comprise bus 902.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 904 of computer system 900 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 900 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 900, whereby processor 904 would execute the analyses and determinations provided by these engines, subject to instructions provided by any one of, or a combination of, the memory components RAM 906, ROM 908, or storage device 910 and user input provided via input device 914.

### Kits for Determining a Kinetic Parameter

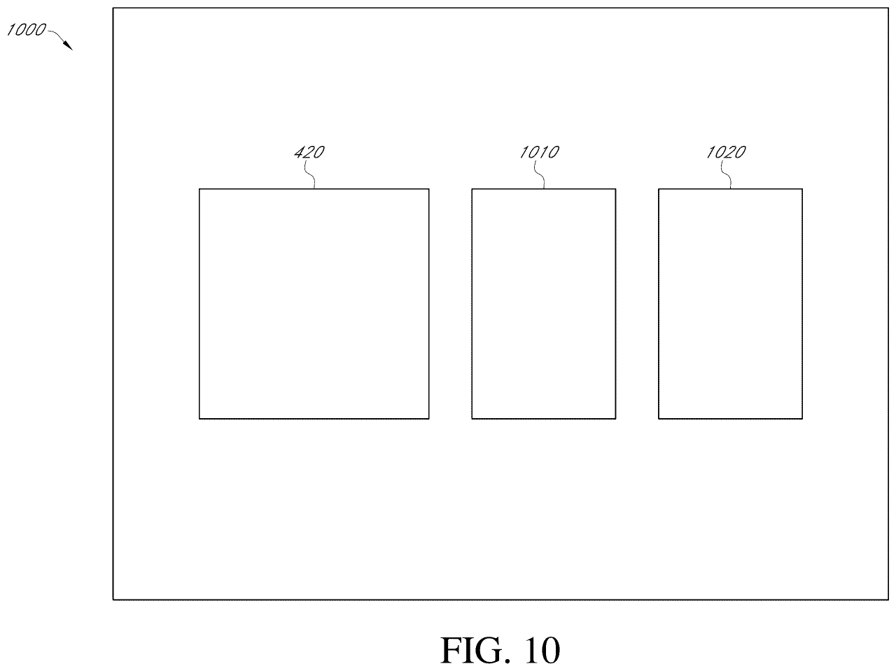

FIG. 10 is a block diagram of a kit 1000 for estimating a kinetic parameter. In various embodiments, the kit comprises an assay module. The assay module may comprise any of assay modules 420, 500, or 600 described herein with respect to FIGS. 4, 5, and 6, respectively. The kit may further comprise a first solution 1010 comprising a plurality of target molecules, such as any plurality of target molecules described herein. The kit may further comprise a second solution 1020 comprising a plurality of competitive inhibitor molecules, such as any plurality of competitive inhibitor molecules described herein.

### EXAMPLES

### Example 1: Experimental Methods

Assays were conducted using a Biacore 5200 (GE Healthcare Bio-Sciences AB, SE-751 84, Uppsala, Sweden) with analysis temperature set to 20° C. All reagent coupling kits and sensors were from GE Healthcare. A biotinylated-avi-tagged 22 kDa target protein was expressed recombinantly and purified in-house using standard protocols. The probe molecule (M_{R}=737 Da) was a PEGylated compound with moderate affinity for the target molecule, where a terminal primary amine on the PEG linker allowed coupling to a CMS sensor chip through EDC/NHS covalent linkage chemistry. All experiments were performed using an assay buffer containing 50 mM 4-(2-hydroxyethyl)-1-piperazineethanesulfonic acid, (HEPES), pH 7.5, containing 0.15 M sodium chloride and 0.2 mM tris(2-carboxyethyl)phosphine (TCEP), and 0.1% polyethyleneglycol Mₙ˜4 kDa), 1 mg/ml CM dextran (Mₙ˜10 kDa). Kinetics of probe binding to tethered target: The target was captured onto a series S SA sensor chip. The soluble probe was injected for 30 s in duplicate over a serial doubling dilution range from 1 μM to 31 nM prepared in assay buffer. The binding curves were double referenced and fitted to a two-compartment 1:1 interaction model for estimation of kₐ and k_{d}. Kinetics of target binding to tethered probe: Channels 2 and 4 of a series S CMS sensor chip were activated in-situ for 8 min using standard EDC/NHS. The chip was undocked, rinsed with buffer and 40 μl of probe solution containing 1 mM probe molecules, diluted in a 1:1 (v/v) solution of DMSO:1 M HEPES (pH 7.5), was pipetted onto the sensing surface and incubated for 2 h at room temperature. The surface was rinsed in buffer and blocked with 1 M ethanolamine for 10 min. The chip surface was rinsed with 100% DMSO, 20 mM NaOH, and ultrapure water, dried, and redocked. Target protein diluted in assay buffer was injected for 60 s in duplicate over a serial-doubling dilution range from 1 μM to 31 nM. For rebinding assay curves: A fresh probe-coated CMS sensor chip was prepared, using a 20 min probe-solution contact time, which lowered the Rₘₐₓ relative to the previous 2 h exposure. 500 nM target was injected over channel 2 (probe-coated) at 30 μL/min for 60 s followed by dissociation. This was repeated (8 replicates) and the target molecule was allowed dissociate from the surface between replicate cycles. An inhibitor injection commenced for each of the target molecule loading injections when the dissociation response decreased to 118 RU. The inhibitor was injected over channels 1 and 2, in duplicate, at 0 μM, 0.016 μM, 0.6 μM, and 4 μM with assay buffer as diluent.

### Example 2: Computational Modeling Methods

Comsol Multiphysics 5.1 (COMSOL AB, Tegnérgatan 23, SE-111 40, Stockholm, Sweden) was used to perform all numerical simulations. A computational model replicating the systems and methods described herein was created. Microfluidic channels employed in biosensors may have high aspect ratios where side walls are far apart relative to the top and bottom walls, allowing microchannel width to be neglected and reducing the model to a cross-section through the microchannel. A two-dimensional flow cell geometry with height h=20 μM and length l=0.5 mm housing a hydrogel film grafted to the flow cell sensing region (i.e., 200 nm×0.5 mm sensing domain) was meshed with >14 k elements. This mesh was optimized until no detectable change was observed in the simulation output and included a higher density of elements at the hydrogel interfacial boundaries, arriving at 6762 elements over the hydrogel domain. The incompressible form of the Navier-Stokes equation (with p being the density, p being the pressure, and μ being the dynamic viscosity) was used to solve the two-dimensional velocity profile u through the channel:

ρu·∇u=−∇p+μ∇²u (32)

Steady-state conditions, constant flow rate, and atmospheric pressure were assumed. The velocity at the walls was set to zero and the inlet velocity was variable. The velocity vector field was then solved over the full domain. The flow velocity vector field was coupled to the steady-state advection and diffusion equation for a dilute species to solve for the analyte concentration field in the bulk flow (with D being the diffusion coefficient, c being the analyte concentration, and R being a reaction term:

∇·(−D∇c)+u·Δc=R (33)

Initially the analyte concentration in the microchannel was c=0. At the inlet the initial analyte concentration profile along the microchannel height was defined by multiplying the concentration by a rectangular function to produce a rectangular pulse of injected sample for a given contact time. Target molecules were assumed to be distributed within the hydrogel. The numerical simulation does not account for transport, as that is a manifestation of the interplay of surface reactions and advection/diffusion, which is solved numerically. Thus, only the surface reactions given by Equations (34)-(38) are included and coupled to the advection and diffusion equation.

$$\frac{d [ A ]}{dt} = - {k}_{a} * [ A ] * [ B ] + {k}_{d} * [ AB ] \qquad ( 34 )$$

$$\frac{d [ B ]}{dt} = - {k}_{a} * [ A ] * [ B ] + {k}_{d} * [ A B ] - {k}_{a}^{′} * [ B ] * [ P ] + {k}_{d}^{′} * [ B P ] \qquad ( 35 )$$

$$\frac{d [ A B ]}{dt} = {k}_{a} * [ A ] * [ B ] - {k}_{d} * [ AB ] \qquad ( 36 )$$

$$\frac{d [ P ]}{dt} = - {k}_{a}^{′} * [ B ] * [ P ] + {{k}_{d}}^{′} * [ B P ] \qquad ( 37 )$$

$$\frac{d [ B P ]}{dt} = {k}_{a}^{′} * [ B ] * [ P ] - {{k}_{d}}^{′} * [ B P ] \qquad ( 38 )$$

For formation of an irreversible complex (AB*), Equations (39)-(44) were used in place of Equations (1)-(4).

$$\frac{d [ A ]}{dt} = - {k}_{a} * [ A ] * [ B ] + {k}_{d} * [ AB ] \qquad ( 39 )$$

$$\frac{d [ B ]}{dt} = - {k}_{a} * [ A ] * [ B ] + {k}_{d} * [ A B ] - {{k}_{a}}^{′} * [ B ] * [ P ] + {{k}_{d}}^{′} * [ B P ] \qquad ( 40 )$$

$$\frac{d [ A B ]}{dt} = {k}_{a} * [ A ] * [ B ] - {k}_{d} [ A B ] - {k}_{inact} [ AB ] \qquad ( 41 )$$

$$\frac{d [ P ]}{dt} = - {k}_{a}^{′} * [ B ] * [ P ] + {{k}_{d}}^{′} * [ B P ] \qquad ( 42 )$$

$$\frac{d [ B P ]}{dt} = {k}_{a}^{′} * [ B ] * [ P ] - {{k}_{d}}^{′} * [ B P ] \qquad ( 43 )$$

$$\frac{d [ A B * ]}{dt} = {k}_{inact} * [ AB ] \qquad ( 44 )$$

The time-dependent change in analyte accumulation was found from a surface flux balance at the sensing surface where the simulation was performed in time-stepping mode. The accumulation of affinity complex was expressed in terms of an equivalent biosensor response

$$R = \frac{{c}_{s}}{{c}_{max}} * {M}_{r} * 1 {0}^{6} .$$

The following parameters were held fixed: R₀=0.1Rₘₐₓ, [A]=1 mM, kₐ=1×10 6 M⁻¹s⁻¹, k_{d}=0.001 s⁻¹, k′ₐ=1×10⁷ M⁻¹s⁻¹, k_{d}′=0.05 s⁻¹, k_{inact}=1 s⁻¹, M_{r_B}=30 kDa, M_{r_A}=200 kDa, [P]=1 mM (equivalent to 30,000R U when fully saturated by B).

A 200 nm thick hydrogel was modeled as a volume containing a homogenous density of hydrogel grafted to the sensing surface that decreases rapidly at the hydrogel-liquid interface according to the hydrogel density function density=1−exp(−100*(1−z)), where z is the unitless relative height of the hydrogel. The concentration of P was assumed to be scaled by the hydrogel density and the diffusion coefficient of all species tethered to the hydrogel (i.e., P, BP) was assumed to be zero. Diffusion of all species inside hydrogel was assumed to be 2-fold lower due to a 2-fold increase in viscosity within the hydrogel relative to the bulk liquid. Soluble species were subject to molecular weight-dependent partitioning. Therefore, parameters related to mass transport of soluble species inside the hydrogel were defined as follows: Diffusion coefficient of A=D_{A}=5×10⁻¹⁰ (m²/s), diffusion coefficient of B=D_{B}=D_{A}(M_{r_B}/M_{r_A})^{1/3,} diffusion coefficient of A inside hydrogel=D_{gel,A}=2*D_{A}*K_{part,A}, where the hydrogel partition coefficient for A=K_{part,A}=exp(10⁻³*M_{r_A}^{2/3}), diffusion coefficient of all soluble species containing B (i.e. B, AB, AB*) inside hydrogel=D_{gel,B}=2*D_{B}*K_{part,B}, where the hydrogel partition coefficient for B=Kₚₐᵣₜ,B=exp(10⁻³*M_{r_B}^{2/3}). The initial conditions for the simulation included addition of tethered P, some fraction of which was in the form of affinity complex BP before the onset of the inhibitor injection. The inhibitor injection was simulated as a sample pulse entering from one end of the rectangular flow cell and exiting at the opposite end. Tethered hydrogel film was located at one of the flow cell walls and was parallel to the direction of flow.

The coupled differential equations given by Equations (12)-(20) (or Equations (21)-(31) for irreversible complexes) were solved numerically coupled to the Navier-Stokes and advection/diffusion equations that govern flow, advection, diffusion, and reaction. The simulations assumed a 20 μM thick flow cell housing a sensing region containing a hydrogel film functionalized with P. The entire geometry was discretized in space and solved over incremental time periods to generate surrogate experimental data.

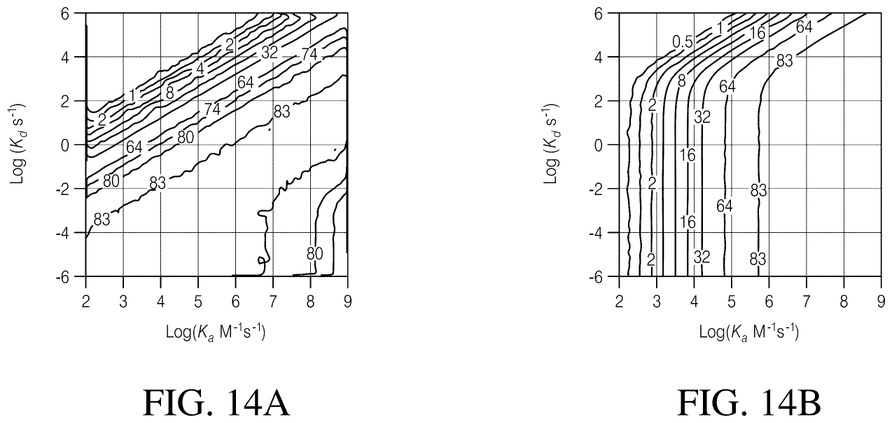

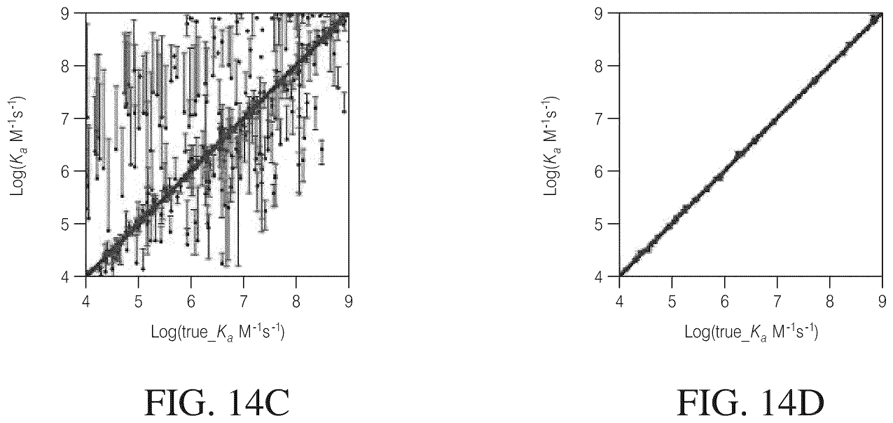

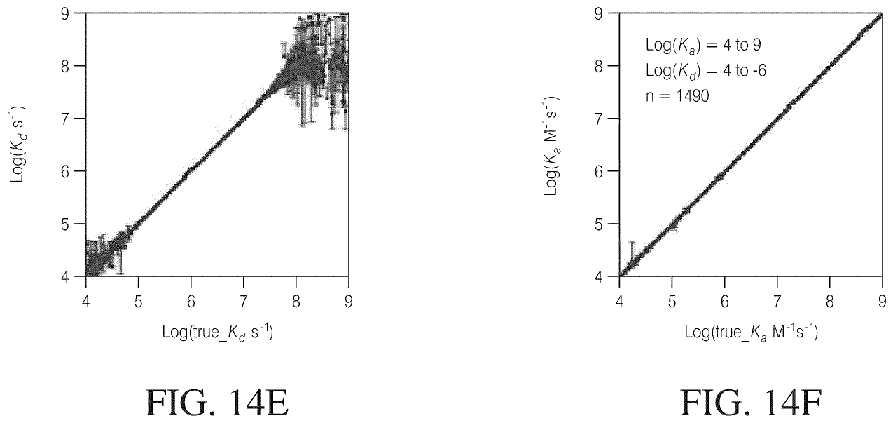

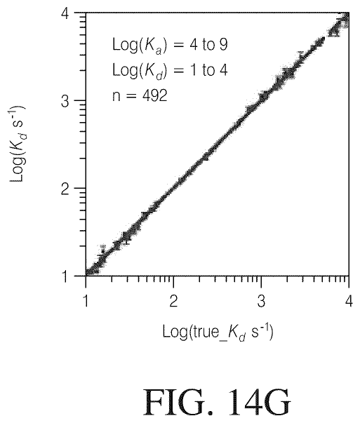

Microsoft Excel and Biaevaluation (GE Healthcare Bio-Sciences AB) were employed for data processing. Graphpad Prism version 6 (GraphPad Software, Inc. 7825 Fay Avenue, Suite 230, La Jolla, CA, 92037, USA) was employed for all plots other than those shown in FIGS. 14A-B. Statistical parameters such as the standard error of the fit (SE) associated with a given parameter returned in the fit were used to report confidence in parameter estimates. The SE is a measure of the information content of the data and specifies the degree to which the curves define the parameter value from the fit. Values <5% indicate high confidence and values >10% indicate that the parameter is poorly defined. The goodness of fit between a model curve and an experimental curve is described by χ² when the number of data points is high and by a regression coefficient R² when the number of values is low. % χ² is the square of the averaged residual response difference expressed as a percentage of maximum response recorded for the curve set. Typically, high quality fits will produce χ² values <5%. Occasionally χ² may be within acceptable limits but the fit may remain questionable if residuals are not distributed randomly. Curves generated by numerical simulation follow deterministic algorithms and reproduce without error and therefore do not require replicates.

All fitted parameters were constrained to a single global value over the entire curve set, providing a more robust fit to the model. The resulting parameter estimates therefore represent the sum of multiple replicated measurements.

### Example 3: Computational Estimation of kₐ for Non-Transient Inhibitors

When analyzing non-transient binders, it can be assumed that k_{d}<<kₜ and therefore that ƒ≅1. The kinetics of BP formation may be predetermined by conventional direct binding kinetics, at low surface density of probe, prior to characterizing inhibitors. These kinetic constants may then be held constant when analyzing dose-dependent inhibition curves, allowing kₐ, k_{d}, and kₜ to be determined by global fitting of Equations (6)-(11). A zero-inhibition curve, where [A]=0, may be included to allow kₜ to be estimated in the absence of inhibition. Pre-estimation of kₐ′ and k_{d}′ by conventional direct binding kinetics may be avoided by testing the soluble probe as an inhibitor sample while also maintaining it tethered as the surface-bound probe. In this case, AB may become a fully soluble form of BP and hence both kₐ′ and kₐ may govern the same interaction but in reverse format, but may be related through molecular weight (Mᵣ)-dependent diffusion scaling, where kₐ′≅kₐ*(M_{r_P}/M_{r_B})^{1/3}. In such case, substitution into Equation (7) may allow kₐ′, k_{d}′, and kₜ to be estimated from globally fitting Equations (6)-(11). These parameters may then be held constant when fitting unmodified Equation (7) to the remainder of the inhibitor panel for estimation of kₐ (and, where appropriate, k_{d}′).

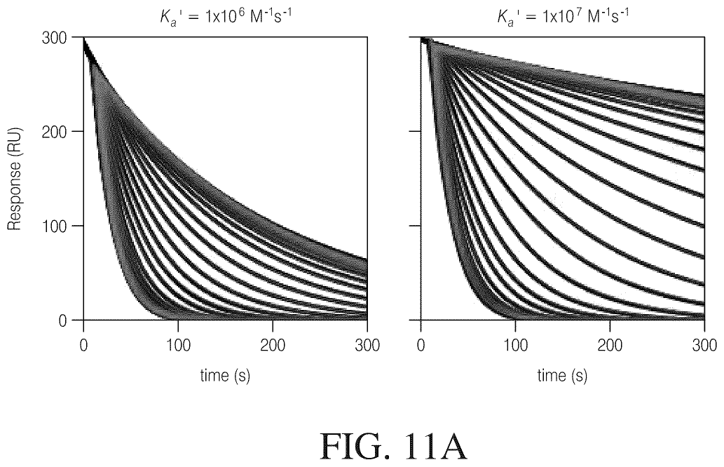

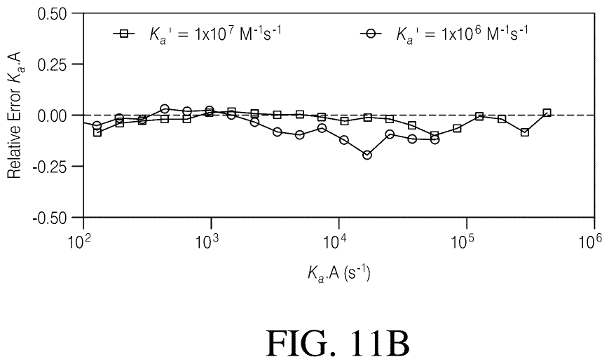

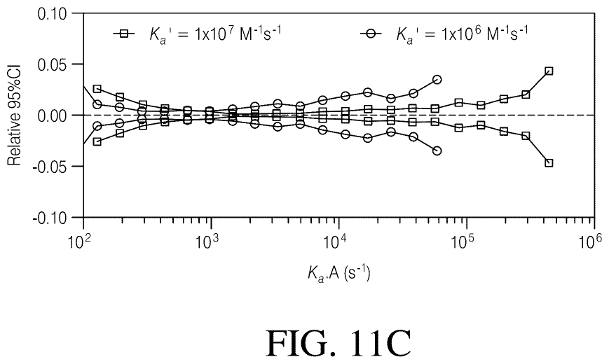

Surrogate experimental data was generated over a wide range in kₐ at a fixed [A] in order to determine the relative error and confidence intervals associated with kₐ estimation for non-transient inhibitors (where k_{d}<<kₜ). The data are shown in FIGS. 11A-C. For the two curve sets in FIG. 11A, the upper and lower limit curves correspond to k_{off} at zero inhibition and k_{d}′ at full inhibition, respectively. These limits define a 2-fold wider response widow for a 10-fold higher kₐ′. Both the relative error (FIG. 11B) and confidence intervals (FIG. 11C) reflect this trend indicating a ˜10-fold increase in measuring range at the higher kₐ′. For FIG. 11A, inhibition curves were fit to Equations (6)-(11), where kₐ was fit locally, giving independent estimates of kₐ for each curve, while all other parameters were held constant.

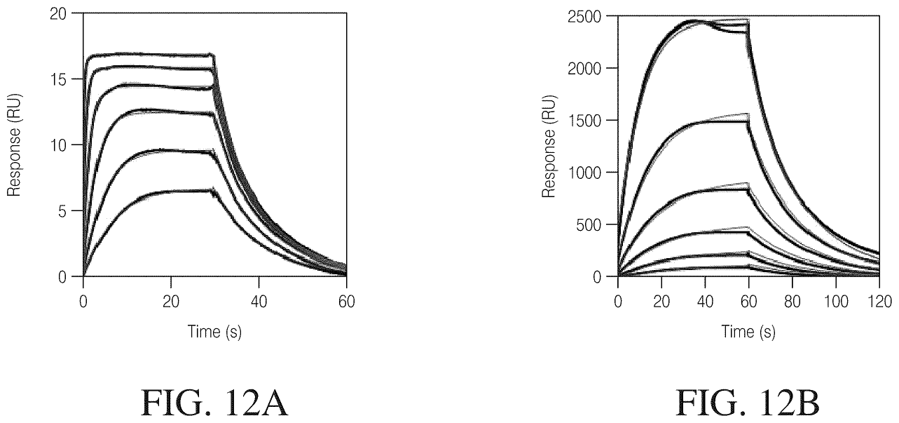

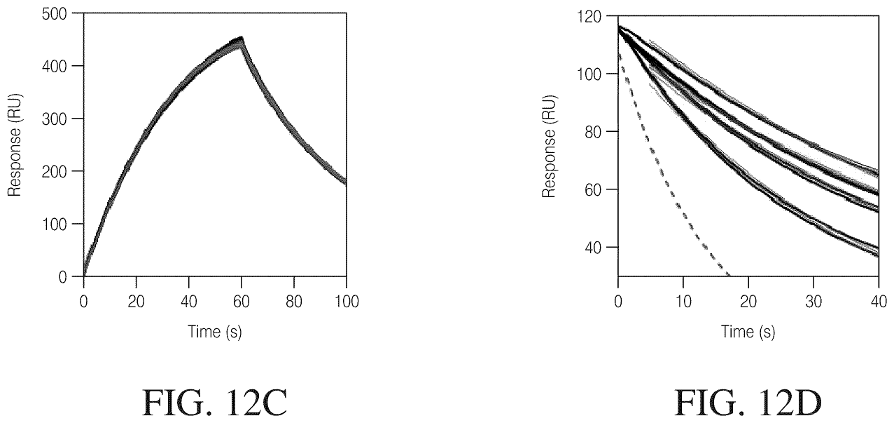

A soluble probe was employed as a surrogate test inhibitor in order to cross-validate parameter return. The experimental data is shown in FIGS. 12A-D. FIG. 12A shows conventional SPR curves for interaction of soluble probe molecules with hydrogel-bound target molecules (Mᵣ=22 kDa). The curves were fitted to a two compartment 1:1 interaction model for estimation of kₐ and k_{d}. Probe molecules (Mᵣ=737 Da) were injected for 30 s in duplicate over a serial doubling dilution range from 1 μM to 31 nM. All parameters were fit globally returning values of kₐ=2.76±0.008 (×10⁶) M⁻¹s⁻¹, k_{d}=0.125±0.0003 s⁻¹, Rₘₐₓ=16.8±0.01 RU and % χ²=0.13. FIG. 12B shows conventional SPR curves for interaction of soluble target molecules with hydrogel-bound probe molecules. The curves were fitted to a two-compartment 1:1 interaction model for estimation of kₐ′ and k_{d}′. Target molecules were injected for 60 s in duplicate over a serial-doubling dilution range from 1 μM to 31 nM. All parameters were fit globally returning values of kₐ′=8.5±0.004 (×10⁴) M⁻¹s⁻¹, k_{d}′=0.075 s⁻¹, Rₘₐₓ=4052±0.001 RU and % χ²=0.31. FIG. 12C shows estimation of Rₘₐₓ from the target capture region of the rebinding assay curves. 500 nM target was injected over a probe molecule-coated surface at 30 μL/min for 60 s followed by dissociation, where the inhibitor injection was performed when the response had decreased to ˜118 RU (this region was excluded to facilitate estimation of Rₘₐₓ). A kinetic two-compartment model was fit to 8 replicate curves over the region shown in the plot, neglecting dissociation beyond 100 s, which contained the inhibition region of the curves returning an average Rₘₐₓ=1025±10 RU. FIG. 12D shows inhibition curves for the rebinding assay of FIG. 12C. The curves are shown time normalized to the point at which the dissociation response was <118 RU and were fit to Equations (6)-(11) for estimation of kₐ. The 8 replicate curves in FIG. 12C correspond to target loading for duplicate injections of inhibitor at 0 μM, 0.016 μM, 0.6 μM and 4 μM, resulting in dose-dependent inhibition regions that were then fit to Equations (6)-(11). This returned values of kₐ=3.07±0.01 (×10⁶) M⁻¹s⁻¹, kₜ=10.55 s⁻¹, and % χ²=1.1, where kₐ′, k_{d}′, k_{d}, and Rₘₐₓ were held constant at the values estimated from FIGS. 12A-C. R₀ was fit locally and both kₐ and kₜ were fit globally. Higher inhibitor concentrations were prevented by solubility limitations.

Direct binding kinetics returned kₐ=2.76±0.008 (×10⁶) M⁻¹s⁻¹ for soluble-probe binding to immobilized-target (FIG. 12A). After diffusion re-scaling this became kₐ=1.0×10⁵ M⁻¹s⁻¹. This is within 15% of kₐ′=8.5±0.004 (×10⁴) M⁻¹s⁻¹, which was obtained for the reverse format (FIG. 12B), where soluble target molecules were bound to immobilized probe molecules. More importantly, kₐ=2.76±0.008 (×10⁶) M⁻¹s⁻¹ for direct binding of probe (FIG. 12C) was within 10% of kₐ=3.07±0.01 (×10⁶) M⁻¹s⁻¹)) returned from inhibition of rebinding (FIG. 12D) thereby cross-validating the results between these assay formats. The assay may thus be compatible with a throughput of more than 500 inhibitors/day using a Biacore 8K SPR system, assuming four concentrations per inhibitor. However, the assay may also be conducted in singleton when screening, allowing medium-sized compound collections (such as fragment libraries) to be analyzed in a single run.

### Example 4: Computational Estimation of Transient Kinetics

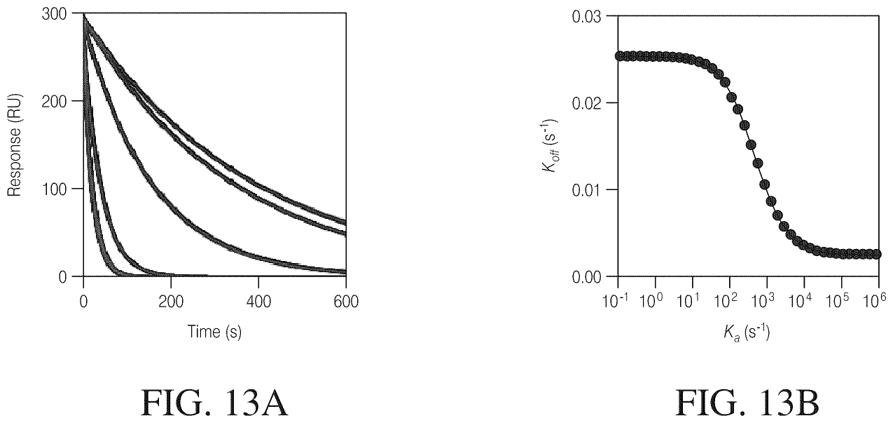

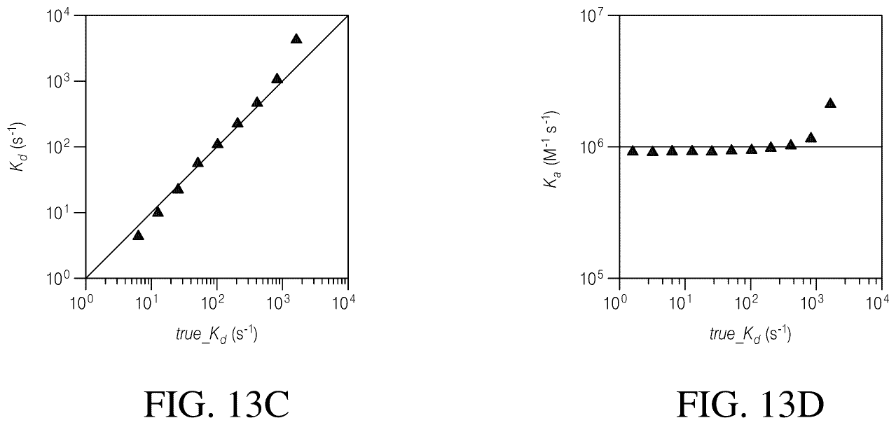

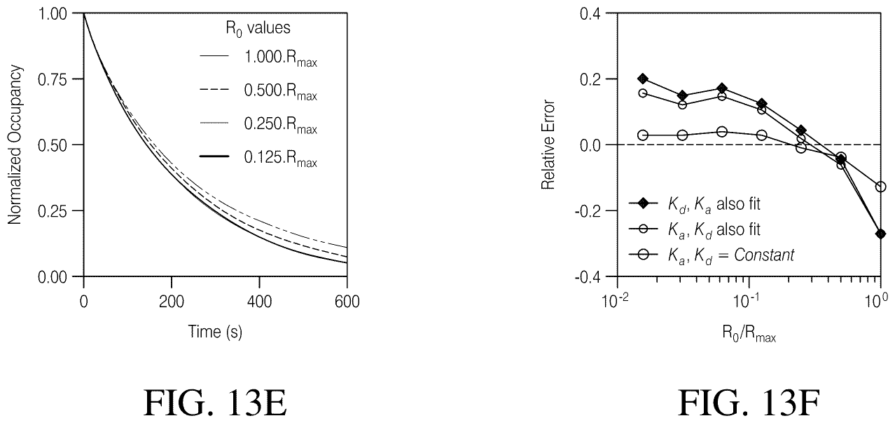

FIGS. 13A-F show kinetic detection limits for transient inhibitor binding. FIG. 13A shows inhibition curves corresponding to kₐ values of 10⁴, 10⁵, 10⁶, 10⁷, 10⁸ and 10⁹ M⁻¹s⁻¹, each with 8 superimposable replicates corresponding to k_{d} (0, 0.1, 1, and 10 s⁻¹) all with, and without, inclusion of an irreversible adduct formation rate k_{inact} (0, 1 s⁻¹). The degree of inhibition increased with increasing kₐ and the inhibition curves superimposed at full inhibition, corresponding to the two highest kₐ values of 10⁸ and 10⁹. FIG. 13B shows partition curves for loss of inhibition of rebinding as a function of transient k_{d}, where the apparent dissociation rate constant k_{off} was obtained by fitting Equation (6) to surrogate experimental data over a range in k_{d} at kₐ=1×10⁶ M⁻¹ s⁻¹. FIGS. 13C and 13D show correlation of fitted kinetic constants versus the corresponding true values used in generating the parent surrogate data, where both kinetic constants were constrained as global values for each curve set. Each curve set contained three replicate injections of 1 mM A, performed at flow rates of 0.1 m/s, 0.01 m/s, 0.001 m/s, respectively. kₜ was determined at each flow rate using blank inhibition curves, where [A]=0, and was then held constant for estimation of kinetics. FIG. 13E shows divergence of response-normalized dissociation curves over a range in R₀/Rₘₐₓ, where [A]=0. FIG. 13F shows relative error in kinetic parameters returned from fitting Equations (6)-(11) to surrogate experimental data over a wide range in R₀/Rₘₐₓ.

Inhibition of rebinding is maintained even when k_{d}>>kₜ in cases when the inhibition complex is stabilized by rapid alkylation. Such rapid alkylation may permanently inactivate the inhibition complex where k_{inact}>k_{d} and k_{inact}>kₜ and would lead to partitioning according to z=1+k_{inact}/kₜ. Such high k_{inact} values may be unfavorable in drug discovery due to their non-specific alkylation potential. Thus, we may consider only k_{inact}<1 s⁻¹, allowing z to be neglected, since kₜ>>1 s⁻¹. Therefore, estimation of kₐ and k_{d} for transient irreversible binding may remain identical to that for reversible inhibitors. To illustrate this, an irreversible inhibition complex (AB*) was added to the coupled differential equations of the computational model such that formation of an irreversible inhibition complex (AB*) proceeded at a rate constant k_{inact}, with d[AB*]/dt=k_{inact} [AB]. Inhibition curves corresponding to six kₐ values were replicated eight times, at four transient binding levels (0.1≤k_{d}≤10 s⁻¹) with, and without, inclusion of irreversible alkylation (k_{inact}=1 s⁻¹) for a total of forty-eight separate conditions. The forty-eight inhibition curves superimpose well at each kₐ value, as shown in FIG. 13A, indicating that the rebinding assay may be expected to return kₐ estimates using Equations (6)-(11), without inclusion of partition functions (i.e. ƒ=1) to correct the flux balance when kₜ>>k_{d} or kₜ>>k_{inact}.

As shown in FIG. 13B, inhibition for transient inhibitors will decrease when kₜ is on the same order as, or less than, k_{d}, and follows a partition function ƒ=1/(1+k_{d}/kₜ), where the total hydrogel escape flux becomes β=kₜ+ƒ*kₐ*[A]. This implies that the measurable range for k_{d} may be increased by employing conditions that promote a wider range in kₜ, such as wide flow rate variation, lower density of probe molecules, and modulating secondary transient interactions (e.g., affinity, electrostatic, or metal chelate) between the hydrogel and the target. Equation (9) assumes exponential dissociation/inhibition curves while multiphasic dissociation curves are expected at high Dₐ values and are readily observable for Dₐ=100. Nevertheless, this approximation remains valid despite relatively high Dₐ by constraining the occupancy rage over which dissociation occurs and thereby eliminating the faster early dissociation phase where rebinding is less probable when fewer free sites are available. For example, for the surrogate experimental data in FIG. 13C-D, assume a dissociation phase beginning at 10% of saturation (in other words R₀=0.1Rₘₐₓ) and low systematic error (<5%) over 2 orders for simultaneous estimation of kₐ and k_{d}. Higher R₀/Rₘₐₓ results in higher measurement error, as shown in FIGS. 13E-F. Multiple dissociation phase curves were generated over a range in R₀/Rₘₐₓ, were response-normalized (FIG. 13E), and show a maximum divergence of ˜6% occupancy at R₀/Rₘₐₓ=1 with negligible divergence for R₀/Rₘₐₓ≤0.25. Propagation of this systematic deviation into error in kinetic parameter return was evaluated by fitting Equations (6)-(11) to surrogate experimental data over a wide range in R₀/Rₘₐₓ and is shown in FIG. 13F. The lowest systematic error was observed for estimation of kₐ when k_{d} was held constant, resulting in a maximum of 13% underestimation at R₀/Rₘₐₓ=1, while <4% error was observed at R₀/Rₘₐₓ<0.2. This error may be related to divergence of the dissociation curves, when R₀/Rₘₐₓ>0.2, causing a further ˜2-fold increase in systematic deviation when both kₐ and k_{d} were fit simultaneously. Spatial concentration gradients may exist within the hydrogel in such transient time regimes and may contribute to the observed error. Fitting the full ODE based model given by Equations (12)-(20) may reduce this error, as numerically solving these differential equations may not require making simplifying steady-state assumptions which are implicit in the phenomenological model given by Equations (6)-(11).

### Example 5: Limit of Detection and Parameter Return Accuracy

FIGS. 14A-G show comparisons of a competitive kinetics assay with the rebinding assays described herein in terms of sensitivity and parameter estimation, evaluated using Monte Carlo simulations seeded with pseudo-random kinetic values. FIG. 14A shows an affinity space plot for a competitive kinetics assay with contour curves connecting regions of equal response (response value (RU) is given inset on contour curves). FIG. 14B shows an affinity plot space for the rebinding assay described herein. FIG. 14C shows correlation of true kₐ with kₐ estimated from fitting the competitive kinetic model, for pseudo-random kₐ/k_{d} combinations spanning 10 orders of magnitude with limits 4≤log kₐ≤9 and −6≤log k_{d}≤4. The diagonal, or unit slope, indicates the accuracy of parameter return and the standard error associated with the parameter fit is indicated by the ±SE bars. FIG. 14D shows the correlation of true kₐ with kₐ estimated from fitting the competitive kinetic model with transient k_{d} values eliminated by restricting the k_{d} limit to −6≤log k_{d}≤1. FIG. 14E shows the data of FIG. 14D given in terms of true k_{d} versus fitted k_{d} correlation. FIG. 14F shows the correlation of true kₐ with kₐ estimated from fitting the rebinding model described herein. Assay parameters for the Monte Carlo simulations were matched with the parameters using in the competitive kinetics assay model and are shown over the full kinetic range given by limits of 4≤log kₐ≤9 and −6≤log k_{d}≤4. FIG. 14G shows correlation of true k_{d} versus k_{d} estimated from the rebinding model described herein, with transient k_{d} values eliminated by restricting the k_{d} limit to −6≤log k_{d}≤1.

Monte Carlo simulations seeded with pseudo-random kinetic values were generated to compare solution-phase competitive binding kinetics with the rebinding assay. For each assay format, the iso-response contour on the top left-hand corner define the sensitivity limit and indicate a broad measuring range. For competitive kinetics shown in FIG. 14A, the iso-response contours are affinity isotherms over the majority of the kinetic range as they follow a unit slope while vertical iso-response contours (which indicate kₐ-driven inhibition) are confined to tightly bound inhibitors on the bottom right-hand corner. Conversely, vertical iso-response contours dominate for the rebinding assay shown in FIG. 14B, implying kₐ-driven inhibition, while iso-response contours of unit slope 1 are confined to the extreme transient kinetic region because such transient complexes approach steady-state before exiting the hydrogel via diffusive or convective mass transport.

The kinetic measuring range of both formats were evaluated using Monte Carlo simulations, where each respective kinetic model was backfit to a large set of simulated curve sets produced from each respective parent model and is shown in FIGS. 14C-G. For competitive kinetics, the kₐ-correlation plot in FIG. 14C shows that kinetic parameters are poorly defined over a broad range in kₐ when k_{d} is transient, while the remainder of the simulations returned reliable kₐ estimates, as shown in FIG. 14D. Furthermore, the k_{d}-correlation plot in FIG. 14E also shows poor k_{d} estimates for transient binders. In contrast, the kₐ-correlation plot for the rebinding assay shown in FIG. 14F indicates that kₐ remains well defined over the full range of kₐ and included highly transient binders. In addition, the associated k_{d}-correlation plot shown in FIG. 14G indicates that reliable k_{d} estimates are returned for transient binders, consistent with the results obtained for surrogate data generated from the full computational model and fitted to Equations (6)-(11).

In describing the various embodiments, the specification may have presented a method or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments. Similarly, any of the various system embodiments may have been presented as a group of particular components. However, these systems should not be limited to the particular set of components, now their specific configuration, communication and physical orientation with respect to each other. One skilled in the art should readily appreciate that these components can have various configurations and physical orientations (e.g., wholly separate components, units and subunits of groups of components, different communication regimes between components).

Although specific embodiments and applications of the disclosure have been described in this specification, these embodiments and applications are exemplary only, and many variations are possible.

### RECITATION OF EMBODIMENTS

Embodiment 1. An assay method for estimating a kinetic parameter, comprising:

introducing a plurality of target molecules to a first location, the first location comprising: (i) a first surface, (ii) a matrix bound to the first surface, and (iii) a plurality of probe molecules bound to the matrix;

monitoring, via a detection method, a first amount of an intermolecular complex generated by the plurality of probe molecules and the plurality of target molecules;

when the first amount exceeds a threshold value, stopping the introduction of the plurality of target molecules and introducing a plurality of competitive inhibitor molecules to the first location;

monitoring, via the detection method, a second amount of the intermolecular complex; and

estimating the kinetic parameter based upon the second amount.

Embodiment 2. The assay method of Embodiment 1, wherein the kinetic parameter comprises an association rate constant between the plurality of target molecules and the plurality of competitive inhibitor molecules.

Embodiment 3. The assay method of Embodiment 1 or 2, wherein the kinetic parameter comprises a dissociation rate constant between the plurality of target molecules and the plurality of competitive inhibitor molecules.

Embodiment 4. The assay method of any one of Embodiments 1-3, wherein the first location comprises a first channel in a flow cell.

Embodiment 5. The assay method of Embodiment 4, wherein (a) comprises: starting a flow of the plurality of target molecules through the first channel and wherein (c) comprises: when the first amount exceeds a threshold value, stopping the flow of the plurality of target molecules through the first channel and starting a flow of the plurality of competitive inhibitor molecules through the first channel.

Embodiment 6. The assay method of Embodiment 4 or 5, further comprising monitoring, via the detection method, a second location comprising a second channel in a flow cell, wherein the second location comprises a second surface.

Embodiment 7. The assay method of Embodiment 6, wherein the second location does not comprise a probe molecule bound to at least the second surface.

Embodiment 8. The assay method of any one of Embodiments 1-3, wherein the first location comprises a first well in a well plate.

Embodiment 9. The assay method of Embodiment 8, wherein (a) comprises: placing the plurality of target molecules into the first well and wherein (c) comprises: when the first amount exceeds a threshold value, stopping the placement of the plurality of target molecules into the first well and placing the plurality of competitive inhibitor molecules into the first well.

Embodiment 10. The assay method of Embodiment 8 or 9, further comprising monitoring, via the detection method, a second location comprising a second well in a well plate, wherein the second location comprises (i) a surface and (ii) a matrix bound to the surface.

Embodiment 11. The assay method of Embodiment 10, wherein the second location does not comprise a matrix.

Embodiment 12. The assay method of Embodiment 10 or 11, wherein the second location does not comprise a plurality of probe molecules.

Embodiment 13. The assay method of any one of Embodiments 1-12, wherein the matrix comprises a hydrogel.

Embodiment 14. The assay method of any one of Embodiments 1-13, wherein the kinetic parameter is within a predetermined range and wherein one or more dimensions of the matrix are chosen to sensitize the method to the predetermined range.

Embodiment 15. The assay method of any one of Embodiments 1-14, wherein the one or more dimensions of the matrix are within a range from 5 nanometers (nm) to 1,000 nm.

Embodiment 16. The assay method of any one of Embodiments 1-15, wherein the kinetic parameter is within a predetermined range and wherein a density of the plurality of probe molecules is chosen to sensitize the method to the predetermined range.

Embodiment 17. The assay method of any one of Embodiments 1-16, wherein the detection method comprises a surface plasmon resonance (SPR) method.

Embodiment 18. The assay method of any one of Embodiments 1-17, wherein the first amount of the intermolecular complex is generated from reversible affinity interactions between the plurality of probe molecules and the plurality of target molecules.

Embodiment 19. The assay method of any of Embodiments 1-18, wherein (b) comprises, for a plurality of points in time: detecting, via the detection method, a detection signal indicative of the first amount.

Embodiment 20. The assay method of Embodiment 19, wherein (c) comprises, for a plurality of points in time: (i) comparing the first amount with the threshold value; and (ii) if the first amount exceeds the threshold value, stopping the introduction of the plurality of target molecules and introducing the plurality of competitive inhibitor molecules to the first location.

Embodiment 21. The assay method of Embodiment 18 or 19, wherein the first amount increases over the plurality of points in time.

Embodiment 22. The assay method of any of Embodiments 1-21, wherein (d) comprises, for a plurality of points in time: detecting, via the detection method, a detection signal indicative of the second amount.

Embodiment 23. The assay method of Embodiment 22, wherein the second amount decreases over the plurality of points in time.

Embodiment 24. The assay method of any one of Embodiments 1-23, further comprising, prior to (a), binding the matrix to the first surface and binding the plurality of probe molecules to the matrix.

Embodiment 25. An assay system for estimating a kinetic parameter, comprising:

a flow manifold capable of fluidically coupling to an assay module, the assay module comprising: (i) a first location comprising a first surface, (ii) a matrix bound to the first surface,

and (iii) a plurality of probe molecules bound to the matrix;

a detector; and

a controller configured to:
direct the flow manifold to introduce a first solution comprising the plurality of target molecules to the first location;
direct the detector to monitor a first amount of an intermolecular complex generated by the plurality of probe molecules and the plurality of target molecules;
direct the flow manifold to, when the first amount exceeds a threshold value, stop the introduction of the first solution and introduce a second solution comprising a plurality of competitive inhibitor molecules to the first location;
direct the detector to monitor a second amount of the intermolecular complex; and
determine the kinetic parameter based upon the second amount.

Embodiment 26. The assay system of Embodiment 25, wherein the kinetic parameter comprises an association rate constant between the plurality of target molecules and the plurality of competitive inhibitor molecules.

Embodiment 27. The assay system of Embodiment 25 or 26, wherein the kinetic parameter comprises a dissociation rate constant between the plurality of target molecules and the plurality of competitive inhibitor molecules.

Embodiment 28. The assay system of any one of Embodiments 25-27, wherein the first location comprises a first channel in a flow cell.

Embodiment 29. The assay system of Embodiment 28, wherein (c)(i) comprises: starting a flow of the plurality of target molecules through the first channel and wherein (c)(iii) comprises: when the first amount exceeds a threshold value, stopping the flow of the plurality of target molecules through the first channel and starting a flow of the plurality of competitive inhibitor molecules through the first channel.

Embodiment 30. The assay system of Embodiment 28 or 29, wherein the controller is further configured to direct the detector monitor a second location comprising a second channel in a flow cell, wherein the second location comprises a second surface.

Embodiment 31. The assay system of Embodiment 30, wherein the second location does not comprise a probe molecule bound to at least the second surface.

Embodiment 32. The assay system of any one of Embodiments 25-27, wherein the first location comprises a first well in a well plate.

Embodiment 33. The assay system of Embodiment 32, wherein (c)(i) comprises: placing the plurality of target molecules into the first well and wherein (c)(iii) comprises: when the first amount exceeds a threshold value, stopping the placement of the plurality of target molecules into the first well and placing the plurality of competitive inhibitor molecules into the first well.

Embodiment 34. The assay system of Embodiment 32 or 33, wherein the controller is further configured to direct the detector to monitor a second location comprising a second well in a well plate, wherein the second location comprises (i) a surface and (ii) a matrix bound to the surface.

Embodiment 35. The assay system of Embodiment 34, wherein the second location does not comprise a matrix.

Embodiment 36. The assay system of Embodiment 34 or 35, wherein the second location does not comprise a plurality of probe molecules.

Embodiment 37. The assay system of any one of Embodiments 25-36, wherein the matrix comprises a hydrogel.

Embodiment 38. The assay system of any one of Embodiments 25-37, wherein the kinetic parameter is within a predetermined range and wherein one or more dimensions of the matrix are chosen to sensitize the method to the predetermined range.

Embodiment 39. The assay system of any one of Embodiments 25-38, wherein the one or more dimensions of the matrix are within a range from 5 nm to 1,000 nm.

Embodiment 40. The assay system of any one of Embodiments 25-39, wherein the kinetic parameter is within a predetermined range and wherein a density of the plurality of probe molecules is chosen to sensitize the method to the predetermined range.

Embodiment 41. The assay system of any one of Embodiments 25-40, wherein the detector comprises a surface plasmon resonance (SPR) detector.

Embodiment 42. The assay system of any one of Embodiments 25-41, wherein the first amount of the intermolecular complex is generated from reversible affinity interactions between the plurality of probe molecules and the plurality of target molecules.

Embodiment 43. The assay system of any of Embodiments 25-42, wherein (c)(ii) comprises, for a plurality of points in time: detecting a detection signal indicative of the first amount.

Embodiment 44. The assay system of Embodiment 43, wherein (c)(iii) comprises, for a plurality of points in time: (i) comparing the first amount with the threshold value; and (ii) if the first amount exceeds the threshold value, stopping the introduction of the plurality of target molecules and introducing the plurality of competitive inhibitor molecules to the first location.

Embodiment 45. The assay system of Embodiment 43 or 44, wherein the first amount increases over the plurality of points in time.

Embodiment 46. The assay system of any of Embodiments 25-45, wherein (c)(iv) comprises, for a plurality of points in time: detecting a detection signal indicative of the second amount.

Embodiment 47. The assay system of Embodiment 46, wherein the second amount decreases over the plurality of points in time.

Embodiment 48. An assay module for estimating a kinetic parameter, comprising:
(i) a first location comprising a first surface;
(ii) a matrix bound to the first surface; and
(iii) a plurality of probe molecules bound to the matrix and configured to form an intermolecular complex with a plurality of target molecules,

wherein the target molecules escape the intermolecular complex and the first location at a predetermined rate, the rate based on a thickness of the matrix, a density of the matrix, an extent of crosslinking of the matrix, a viscosity of the matrix, or a density of the plurality of probe molecules.

Embodiment 49. The assay module of Embodiment 48, wherein the first location comprises a first channel in a flow cell.

Embodiment 50. The assay module of Embodiment 48 or 49, further comprising a second location comprising a second channel in a flow cell, wherein the second location comprises a second surface.

Embodiment 51. The assay module of Embodiment 50, wherein the second location does not comprise a probe molecule bound to at least the second surface.

Embodiment 52. The assay module of Embodiment 48, wherein the first location comprises a first well in a well plate.

Embodiment 53. The assay module of Embodiment 52, further comprising a second location comprising a second well in a well plate, wherein the second location comprises (i) a surface and (ii) a matrix bound to the surface.

Embodiment 54. The assay module of Embodiment 53, wherein the second location does not comprise a matrix.

Embodiment 55. The assay module of Embodiment 53 or 54, wherein the second location does not comprise a plurality of probe molecules.

Embodiment 56. The assay module of any one of Embodiments 48-55, wherein the matrix comprises a hydrogel.

Embodiment 57. The assay module of any one of Embodiments 48-56, wherein the one or more dimensions of the matrix are within a range from 5 nm to 1,000 nm.

Embodiment 58. A kit for conducting an assay for estimating a kinetic parameter, comprising:

an assay module comprising a first location comprising (i) a first surface, (ii) a matrix bound to the first surface, and (iii) a plurality of probe molecules bound to the matrix;

a first solution comprising a plurality of target molecules capable of forming a first intermolecular complex with the plurality of probe molecules; and

a second solution comprising a plurality of competitive inhibitor molecules capable of forming a second intermolecular complex with the plurality of target molecules,

wherein the composition of the matrix is optimized for diffusion of the target molecule.

Embodiment 59. The kit of Embodiment 58, wherein the first location comprises a first channel in a flow cell.

Embodiment 60. The kit of Embodiment 58 or 59, wherein the assay module further comprises a second location comprising a second channel in a flow cell, wherein the second location comprises a second surface.

Embodiment 61. The kit of Embodiment 60, wherein the second location does not comprise a probe molecule bound to at least the second surface.

Embodiment 62. The kit of Embodiment 58, wherein the first location comprises a first well in a well plate.

Embodiment 63. The kit of Embodiment 62, wherein the assay module further comprises a second location comprising a second well in a well plate, wherein the second location comprises (i) a surface and (ii) a matrix bound to the surface.

Embodiment 64. The kit of Embodiment 63, wherein the second location does not comprise a matrix.

Embodiment 65. The kit of Embodiment 63 or 64, wherein the second location does not comprise a plurality of probe molecules.

Embodiment 66. The kit of any one of Embodiments 58-65, wherein the matrix comprises a hydrogel.

Embodiment 67. The kit of any one of Embodiments 58-66, wherein the one or more dimensions of the matrix are within a range from 5 nm to 1,000 nm.

Embodiment 68. An assay method for estimating a kinetic parameter, comprising:

introducing a plurality of target molecules to a first location, the first location comprising: (i) a first surface, (ii) a matrix bound to the first surface, and (iii) a plurality of probe molecules bound to the matrix and configured to form an intermolecular complex with the plurality of target molecules;

stopping the introduction of the plurality of target molecules and introducing a plurality of competitive inhibitor molecules to the first location;

monitoring, via a detection method, an amount of the intermolecular complex; and

estimating the kinetic parameter based upon the amount.

Embodiment 69. The assay method of Embodiment 68, wherein the kinetic parameter comprises an association rate constant between the plurality of target molecules and the plurality of competitive inhibitor molecules.

Embodiment 70. The assay method of Embodiment 68 or 69, wherein the kinetic parameter comprises a dissociation rate constant between the plurality of target molecules and the plurality of competitive inhibitor molecules.

Embodiment 71. The assay method of any one of Embodiments 68-70, wherein the first location comprises a first channel in a flow cell.

Embodiment 72. The assay method of Embodiment 71, wherein (a) comprises: starting a flow of the plurality of target molecules through the first channel and wherein (b) comprises: when the amount exceeds a threshold value, stopping the flow of the plurality of target molecules through the first channel and starting a flow of the plurality of competitive inhibitor molecules through the first channel.

Embodiment 73. The assay method of Embodiment 71 or 72, further comprising monitoring, via the detection method, a second location comprising a second channel in a flow cell, wherein the second location comprises a second surface.

Embodiment 74. The assay method of Embodiment 73, wherein the second location does not comprise a probe molecule bound to at least the second surface.

Embodiment 75. The assay method of any one of Embodiments 68-70, wherein the first location comprises a first well in a well plate.

Embodiment 76. The assay method of Embodiment 75, wherein (a) comprises: placing the plurality of target molecules into the first well and wherein (b) comprises: when the amount exceeds a threshold value, stopping the placement of the plurality of target molecules into the first well and placing the plurality of competitive inhibitor molecules into the first well.

Embodiment 77. The assay method of Embodiment 75 or 76, further comprising monitoring, via the detection method, a second location comprising a second well in a well plate, wherein the second location comprises (i) a surface and (ii) a matrix bound to the surface.

Embodiment 78. The assay method of Embodiment 77, wherein the second location does not comprise a matrix.

Embodiment 79. The assay method of Embodiment 77 or 78, wherein the second location does not comprise a plurality of probe molecules.

Embodiment 80. The assay method of any one of Embodiments 68-79, wherein the matrix comprises a hydrogel.

Embodiment 81. The assay method of any one of Embodiments 68-80, wherein the kinetic parameter is within a predetermined range and wherein one or more dimensions of the matrix are chosen to sensitize the method to the predetermined range.

Embodiment 82. The assay method of any one of Embodiments 68-81, wherein the one or more dimensions of the matrix are within a range from 5 nanometers (nm) to 1,000 nm.

Embodiment 83. The assay method of any one of Embodiments 68-82, wherein the kinetic parameter is within a predetermined range and wherein a density of the plurality of probe molecules is chosen to sensitize the method to the predetermined range.

Embodiment 84. The assay method of any one of Embodiments 68-83, wherein the detection method comprises a surface plasmon resonance (SPR) method.

Embodiment 85. The assay method of any one of Embodiments 68-84, wherein the amount of the intermolecular complex is generated from reversible affinity interactions between the plurality of probe molecules and the plurality of target molecules.

Embodiment 86. The assay method of any of Embodiments 68-85, wherein (c) comprises, for a plurality of points in time: detecting, via the detection method, a detection signal indicative of the amount.

Embodiment 87. The assay method of Embodiment 86, wherein (b) comprises, for a plurality of points in time: (i) comparing the amount with the threshold value; and (ii) if the amount exceeds the threshold value, stopping the introduction of the plurality of target molecules and introducing the plurality of competitive inhibitor molecules to the first location.

Embodiment 88. The assay method of Embodiment 86 or 87, wherein the amount increases over the plurality of points in time.

Embodiment 89. The assay method of any one of Embodiments 68-88, further comprising, prior to (a), binding the matrix to the first surface and binding the plurality of probe molecules to the matrix.

What is claimed is:

1. An assay method for estimating a kinetic parameter, comprising:
introducing a plurality of target molecules to a first location, the first location comprising: (i) a first surface, (ii) a matrix bound to the first surface, and (iii) a plurality of probe molecules bound to the matrix;
monitoring, via a detection method, a first amount of an intermolecular complex generated by the plurality of probe molecules and the plurality of target molecules;
when the first amount exceeds a threshold value, stopping an introduction of the plurality of target molecules and introducing a plurality of competitive inhibitor molecules to the first location;
monitoring, via the detection method, a second amount of the intermolecular complex; and
estimating the kinetic parameter based upon the second amount.

2. The assay method of claim 1, wherein the kinetic parameter comprises an association rate constant between the plurality of target molecules and the plurality of competitive inhibitor molecules or a dissociation rate constant between the plurality of target molecules and the plurality of competitive inhibitor molecules.

3. The assay method of claim 1, wherein the first location comprises a first channel in a flow cell or a first well in a well plate.

4. The assay method of claim 3, further comprising monitoring, via the detection method, a second location comprising a second channel in a flow cell or a second well in a well plate, wherein the second location comprises a second surface.

5. The assay method of claim 4, wherein the second location does not comprise a probe molecule bound to at least the second surface, wherein the second location does not comprise a matrix, or wherein the second location does not comprise a plurality of probe molecules.

6. The assay method of claim 1, wherein the matrix comprises a hydrogel.

7. The assay method of claim 1, wherein the kinetic parameter is within a predetermined range and wherein one or more dimensions of the matrix are chosen to sensitize the assay method to the predetermined range.

8. The assay method of claim 7, wherein the one or more dimensions of the matrix are within a range from 5 nanometers (nm) to 1,000 nm.

9. The assay method of claim 7, wherein the kinetic parameter is within a predetermined range and wherein a density of the plurality of probe molecules is chosen to sensitize the assay method to the predetermined range.

10. The assay method of claim 1, wherein the detection method comprises a surface plasmon resonance (SPR) method.

11. The assay method of claim 1, wherein the first amount of the intermolecular complex is generated from reversible affinity interactions between the plurality of probe molecules and the plurality of target molecules.

12. The assay method of claim 1, wherein the first amount increases over a plurality of points in time or wherein the second amount decreases over the plurality of points in time.

13. The assay method of claim 1, further comprising, prior to (a), binding the matrix to the first surface and binding the plurality of probe molecules to the matrix.

14. An assay system for estimating a kinetic parameter, comprising:
a flow manifold capable of fluidically coupling to an assay module, the assay module comprising: (i) a first location comprising a first surface, (ii) a matrix bound to the first surface, and (iii) a plurality of probe molecules bound to the matrix;
a detector; and
a controller configured to:
direct the flow manifold to introduce a first solution comprising a plurality of target molecules to the first location;
direct the detector to monitor a first amount of an intermolecular complex generated by the plurality of probe molecules and the plurality of target molecules;
direct the flow manifold to, when the first amount exceeds a threshold value, stop an introduction of the first solution and introduce a second solution comprising a plurality of competitive inhibitor molecules to the first location;
direct the detector to monitor a second amount of the intermolecular complex; and
determine the kinetic parameter based upon the second amount.

15. An assay module for estimating a kinetic parameter, comprising:
(i) a first location comprising a first surface;
(ii) a matrix bound to the first surface; and
(iii) a plurality of probe molecules bound to the matrix and configured to form an intermolecular complex with a plurality of target molecules,
wherein the target molecules escape the intermolecular complex and the first location at a predetermined rate, the rate based on a thickness of the matrix, a density of the matrix, an extent of crosslinking of the matrix, a viscosity of the matrix, or a density of the plurality of probe molecules.
